SEQUENCE LISTING (6 sequences)

SEQ ID NO: 1 - protein, 547 aa, Mus musculus
MPSGTSQCEDGSAGGFQHLDMHSEKRQLEKGPSGDKDRVWIRPDTPSRCTWQLGRAMADSPHYHTVLTKSPKILPDILRKIGNTPMVRINKISKNAGLKCELLAKCEFFNAGGSVKDRISLRMIEDAERAGNLKPGDTIIEPTSGNTGIGLALAAAVKGYRCIIVMPEKMSMEKVDVLRALGAEIVRTPTNARFDSPESHVGVAWRLKNEIPNSHILDQYRNASNPLAHYDDTAEEILQQCDGKLDMLVASAGTGGTITGIARKLKEKCPGCKIIGVDPEGSILAEPEELNQTEQTAYEVEGIGYDFIPTVLDRAVVDKWFKSNDEDSFAFARMLIAQEGLLCGGSSGSAMAVAVKAARELQEGQRCVVILPDSVRNYMSKFLSDKWMLQKGFMKEELSVKRPWWWRLRVQELSLSAPLTVLPTVTCEDTIAILREKGFDQAPVVNESGAILGMVTLGNMLSSLLAGKVRPSDEVCKVLYKQFKPIHLTDTLGTLSHILEMDHFALVVHEQIQYCSNGMSSKQQMVFGVVTAIDLLNFVAAREQTQT

SEQ ID NO: 2 - DNA, 1644 bp, Mus musculus
  source 1..1644: mol_type = other DNA
atgccttcagggacatcccagtgtgaagatggctctgctgggggcttccagcacttggacatgcactcagaaaagagacaactggagaagggcccctcaggggacaaggatcgagtctggatccggcctgataccccaagcagatgtacctggcagctgggcagggccatggcggactccccacattatcacacagtgctgaccaaatcccccaaaattttaccagatattctgaggaaaattgggaacacccctatggtcagaatcaacaagatctcaaagaatgccggtctcaagtgtgagctcttggccaagtgtgagttcttcaatgcgggtgggagtgtgaaggaccgcatcagccttcggatgatcgaagatgctgagcgagctggaaacttgaagcctggagacactatcattgagccaacttctggcaacacagggatcgggctggctctggctgctgcagtgaagggctatcgctgcattatcgtgatgccggagaagatgagtatggagaaggtggatgtgctgcgggctctgggagccgagattgtgaggacgcccaccaatgccagatttgattcccccgagtcccacgtgggagtggcatggcgactgaagaacgaaatccctaattctcacattctggaccagtaccgcaatgccagcaaccctttggcacactacgatgacaccgccgaggagatcctgcagcagtgtgacgggaagctggatatgctggtggcttcagcaggcacgggtggcaccatcacagggatcgccagaaagctgaaggagaagtgccctggctgtaaaatcatcggtgtcgatcctgaaggctccatccttgcggagcccgaggagctgaaccagacggagcaaacagcctatgaggtggaagggattggctacgacttcatcccgacagtcctggacagggcggtggtggataagtggttcaagagcaacgatgaagattccttcgcctttgcccgcatgctcatcgcacaggaaggactgctatgtggtggaagctctggcagcgccatggctgtggctgtgaaggctgcccgggagctgcaggaagggcagcgctgtgtggtcatcctgcctgactctgtgcggaactacatgtccaagttcctgagtgacaaatggatgctgcagaaaggtttcatgaaagaggagctctcagtgaagaggccctggtggtggcgtctgcgtgttcaagagctgagcctgtcggccccgctgaccgtgttgcccacggtcacctgtgaggacaccatcgccatcctccgggagaagggttttgaccaggcacctgtggtcaacgagtctggggccatcctagggatggtgaccctcgggaacatgctgtcatccctgctggctggaaaggtgcggccatcagacgaagtctgcaaagtcctctacaagcagttcaaaccgatccacctgaccgacacgctgggcacactctctcacatcctggagatggaccacttcgccctggtggtccacgagcagatccaatactgcagcaatggcatgtccagcaagcagcagatggtgtttggggttgtcactgccattgacctgctaaacttcgtggcagcccgtgagcagacccagacatag

SEQ ID NO: 3 - protein, 398 aa, Mus musculus
MQKDASLSGFLPSFQHFATQAIHVGQEPEQWNSRAVVLPISLATTFKQDFPGQSSGFEYSRSGNPTRNCLEKAVAALDGAKHSLAFASGLAATITITHLLKAGDEIICMDEVYGGTNRYFRRVASEFGLKISFVDCSKTKLLEAAITPQTKLVWIETPTNPTLKLADIGACAQIVHKRGDIILVVDNTFMSAYFQRPLALGADICMCSATKYMNGHSDVVMGLVSVNSDDLNSRLRFLQNSLGAVPSPFDCYLCCRGLKTLQVRMEKHFKNGMAVARFLETNPRVEKVVYPGLPSHPQHELAKRQCSGCPGMVSFYIKGALQHAKAFLKNLKLFTLAESLGGYESLAELPAIMTHASVPEKDRATLGINDTLIRLSVGLEDEQDLLEDLDRALKAAHP

SEQ ID NO: 4 - DNA, 1197 bp, Mus musculus
  source 1..1197: mol_type = other DNA
atgcagaaggacgcctctttgagcggcttcctgcctagtttccagcatttcgccactcaggccatccacgtgggacaagagcctgagcaatggaattctcgtgccgtggtgctgcccatttcgttggccaccacatttaagcaggacttcccgggccagtcctcgggttttgaatacagccgctctggaaatccaacaaggaattgcttggaaaaagcagtggctgcgttggatggggcaaagcacagtttggcctttgcatcgggtcttgctgccaccattacgattacccatcttttaaaagcaggagatgaaatcatttgcatggatgaagtgtatggaggcaccaacaggtacttcaggagggtggcatctgaatttggactgaagatttcttttgtagattgttccaaaaccaaattgctagaggcagcgattacaccacaaaccaagcttgtttggatcgaaacacccacaaacccaactttgaagttggctgacattggagcctgcgcacaaattgtccacaaacgtggagacatcattttggttgtagataacaccttcatgtctgcatatttccagagacctttggctctgggtgctgatatttgtatgtgttctgccacaaaatacatgaatggccacagcgatgttgtcatgggtttagtgtctgttaattctgatgacctcaatagtcggcttcgtttcctgcagaattcactaggagcagttccttctccttttgattgttacctctgctgccgaggcctgaagacactgcaggtccggatggagaaacatttcaagaatgggatggcggtggctcgtttcctggagaccaatccccgggtagaaaaggttgtttatcctgggctaccctctcaccctcagcatgagctggccaaacgccagtgctcgggctgcccagggatggtcagtttctacatcaagggtgctctgcagcatgctaaggccttcctcaaaaatctaaagctgtttactctggcagagagcctgggaggatatgagagtctggctgagcttccagcaatcatgacccatgcctctgtgcctgagaaggacagagctaccctcgggatcaatgacacactgatacgactttctgtgggcctagaggatgaacaggaccttcttgaagacctggatcgagctttgaaggcagcgcacccttaa

SEQ ID NO: 5 - protein, 293 aa, Mus musculus
MVDSVYRTRSLGVAAEGLPDQYADGEAARVWQLYIGDTRSRTAEYKAWLLGLLRQHGCHRVLDVACGTGVDSIMLVEEGFSVMSVDASDKMLKYALKERWNRRKEPSFDNWVIEEANWLTLDKDVLSGDGFDAVICLGNSFAHLPDCKGDQSEHRLELKNIASMVRPGGLLVIDHRNYDYILSTGCAPPGKNIYYKSDLTKDITTSVLTVNNKAHMVTLDYTVQVPGTGRDGSPGFSKFRLSYYPHCLASFTELVRAAFGGRCQHSVLGDFKPYKPGQAYVPCYFIHVLKKTD

SEQ ID NO: 6 - DNA, 1088 bp, Mus musculus
  source 1..1088: mol_type = other DNA
ccacgcgtccgggatggtggacagcgtgtaccgtacccgctccctgggggtggcggccgaagggctcccggaccagtatgcagatggggaggccgcacgtgtgtggcagctgtacatcggggacacccgcagccgtaccgcagagtacaaggcgtggttgcttgggctgttgcgccagcacgggtgccacagggtgctggacgtagcctgtggcacaggagtggactccatcatgctggtggaagagggcttcagcgtgatgagcgtggacgccagcgacaagatgctgaaatatgcgcttaaggagcgctggaaccggaggaaagagccatcctttgacaattgggtcattgaagaagccaactggttgacgctggacaaagatgtgctttcaggagatggctttgatgctgtcatctgccttgggaacagttttgctcacttgccagactgcaaaggtgaccagagcgagcaccggctggaactaaagaacattgcaagcatggtgcggcccgggggcctgctggtgatcgaccaccgcaactacgactatatcctcagcacaggctgtgcgcccccggggaagaacatctactataagagtgacctgaccaaggacattacgacgtcagtactgacagtcaacaacaaagcccacatggtaaccctggactacacagtgcaggtgccaggcactggcagagatggctctcctggcttcagtaagttccggctctcttactacccacactgtttggcgtctttcacggagttggtgcgagcagcctttgggggcaggtgccagcacagcgtcctgggtgacttcaagccctacaagcctggccaggcctacgttccctgctacttcatccatgtgctcaagaagacagactgagtttctccggctcccagaagcccatgctcaggcaatggcccctaccctaagaccatcccctaatgcagatattgcatttgggtgcagatgtgggggtcgggcaaacggagtaaacaatacagtctgcattctccaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2025/0304911 A1
(43) Pub. Date: Oct. 2, 2025

(54) CYSTEINE PROTOTROPHY

(71) Applicant: Pfizer Inc., New York, NY (US)

(72) Inventors: Mariah Kaitlyn BRANTLEY, Somerville, MA (US); Laura Jane GREENFIELD, Somerville, MA (US); Bhanu Chandra MULUKUTLA, Lawrence, MA (US); Jeffrey Joseph MITCHELL, Nashua, NH (US)

(21) Appl. No.: 18/834,157

(22) PCT Filed: Jan. 30, 2023
(86) PCT No.: PCT/IB2023/050782

§ 371 Date: Jul. 29, 2024

Related U.S. Application Data
(60) Provisional application No. 63/305,833, filed on Feb. 2, 2022.
(60) Provisional application No. 63/479,059, filed on Jan. 9, 2023.

(51) Int. Cl. C12N 5/071; C07K 16/00; C12N 5/10; C12N 9/10; C12N 9/88

(52) U.S. Cl. CPC C12N 5/0602; C07K 16/00; C12N 5/10; C12N 9/1007; C12N 9/88; C12Y 201/0102; C12Y 402/01022; C12Y 404/01001; C12N 2510/02

(57) ABSTRACT

Provided herein are cells, compositions, and related methods for improved cell growth. The expression of one or more of CBS, CTH, and GNMT genes is increased. In some embodiments, nucleic acid constructs, vectors, host cells and related compositions and methods for generating and selecting cysteine prototroph cells are provided.

### BACKGROUND

The present invention relates to cells having cysteine prototrophy, including methods of making and selecting the cells, and uses thereof. Certain embodiments relate to methods of selecting cells that contain one or more exogenous nucleic acid constructs by selecting cells which exhibit cysteine prototrophy.

In the field of biotechnology, it is frequently desirable to introduce exogenous nucleic acids into a host cell. Exogenous nucleic acids may be introduced into host cells for the purpose of, for example, having the host cell manufacture a polypeptide encoded by the introduced nucleic acid. Polypeptides produced from an exogenous nucleic acid may be permitted to remain in the host cell (e.g. in order to study the activity of the recombinant polypeptide in the cell or to affect one or more biochemical pathways in the cell) or the polypeptides may be isolated from the host cell after production (e.g. when the host cell is being used for producing recombinant proteins which will be used in various downstream applications such as medicines, foods, or industrial components).

An important aspect of the process of generating host cells which contain one or more exogenous nucleic acids of interest is the step of isolating/selecting cells which have successfully received the exogenous nucleic acid(s) of interest. Typically, in processes for introducing an exogenous nucleic acid into a host cell, many cells are exposed to the exogenous nucleic acid, but only a small percentage of the cells exposed to the exogenous nucleic acid ultimately are transfected with the nucleic acid. Furthermore, in situations where the objective is to introduce two or more exogenous nucleic acids into a single host cell, the frequency of such events is even rarer. Accordingly, it is important to be able to easily and efficiently select host cells that have received one or more exogenous nucleic acids of interest.

Various methods are known for selecting cells that have received an exogenous nucleic acid of interest. One of the most common methods is to include as part of an exogenous nucleic acid construct a gene which encodes an enzyme which confers resistance to a particular antibiotic or cellular toxin. In this method, cells that have been exposed to the corresponding exogenous nucleic acid of interest may then be exposed to the corresponding antibiotic or cellular toxin, and only cells which have received the exogenous nucleic acid construct will survive (due their manufacture of the enzyme which confers resistance to the antibiotic or cellular toxin). While this method is effective for the selection of cells that have received an exogenous nucleic acid of interest, it may also be undesirable due to the use of the antibiotic or cellular toxin as a selective pressure.

Another method for selecting cells that have received an exogenous nucleic acid is to include as part of an exogenous nucleic acid construct a gene which encodes an enzyme (e.g. glutamine synthetase or dihydrofolate reductase) which is involved in the production of a molecule necessary for cell growth. In this method, cells that have received an exogenous nucleic acid construct that contains a gene encoding for this type of enzyme can be selected for based on the ability of cells that have received the exogenous nucleic acid construct to grow in a cell culture medium that lacks the corresponding molecule necessary for cell growth (e.g. glutamine in the case of glutamine synthetase or thymidine in the case of dihydrofolate reductase).

However, there is a need for improved and alternative compositions and methods for the isolation and selection of cells that have received an exogenous nucleic acid of interest.

Also needed are methods for methods and compositions for improving the ability of cells to proliferate in cysteine-deficient media, and cells having improved ability to proliferate in cysteine-deficient media.

### SUMMARY

The present disclosure relates to compositions and methods for conferring cysteine prototrophy on cells, and uses for these compositions and methods. For example, provided herein is a method of converting a cell that is a cysteine auxotroph to a cysteine prototroph by the introduction of exogenous copies of the cystathionine beta-synthase (“CBS”) gene and the cystathionase (cystathionine gamma-lyase) (“CTH”) gene into the cell. It is further provided herein that methods and compositions for converting a cysteine auxotroph to a cysteine prototroph may be used to efficiently obtain cells that have received one or more exogenous nucleotide sequences of interest. Accordingly, in some embodiments, compositions and methods provided herein may be used as a cysteine selection marker system.

In some embodiments, provided herein is a method of converting a cell that is a cysteine auxotroph to a cysteine prototroph by increasing the expression of the cystathionine beta-synthase (“CBS”) gene, increasing the expression of the cystathionase (cystathionine gamma-lyase) (“CTH”) gene, and increasing the expression of the glycine N-methyltransferase (“GNMT”) gene in the cell. Optionally, the method may further include decreasing the expression of the methionine synthase (“MTR”) gene in the cell.

In some embodiments, provided herein is a cysteine prototroph cell that has an exogenous cystathionine beta-synthase (“CBS”) gene, an exogenous cystathionase (cystathionine gamma-lyase) (“CTH”) gene, and an exogenous glycine N-methyltransferase (“GNMT”) gene in the cell. Optionally, the cell further has decreased expression of the methionine synthase (“MTR”) gene in the cell, such as by mutation or deletion of the MTR gene or a positive regulatory element thereof.

In embodiments provided herein, expression of a gene (e.g. CBS, CTH, GNMT) may be increased by methods known in the art, such as by introducing one or more exogenous copies of the gene of interest into the cell. Gene expression may also be increased, for example, by upregulating transcription of the endogenous gene in the cell (e.g. by modifying a genetic regulatory element to increase transcription), or by upregulating translation of mRNA of the gene. Similarly, expression of a gene may be decreased by methods known in the art, such as deleting or truncating the endogenous gene, downregulating transcription of the endogenous gene in the cell (e.g. by modifying a regulatory element to decrease transcription), by reducing the translation of mRNA of the gene, or by inhibiting activity of the protein (e.g. by a small molecule inhibitor).

In some embodiments, provided herein is a recombinant nucleic acid construct comprising i) a nucleotide sequence of interest; ii) a CBS gene; and iii) a CTH gene. Optionally, the nucleic acid construct further comprises a recombination target sequence. Optionally, the recombination target sequence is a FLP Recognition Target (“FRT”), lox, or Bxb1-recognized sequence. Optionally, the nucleotide sequence of interest is a first nucleotide sequence of interest, and the recombinant nucleic acid construct further comprises a second nucleotide sequence of interest.

In some embodiments, provided herein is a recombinant nucleic acid construct comprising i) a nucleotide sequence of interest and ii) a CBS gene. Optionally, the nucleic acid construct further comprises a recombination target sequence. Optionally, the recombination target sequence is a FLP Recognition Target (“FRT”), lox, or Bxb1-recognized sequence. Optionally, the nucleotide sequence of interest is a first nucleotide sequence of interest, and the recombinant nucleic acid construct further comprises a second nucleotide sequence of interest.

In some embodiments, provided herein is a recombinant nucleic acid construct comprising i) a nucleotide sequence of interest and ii) a CTH gene. Optionally, the nucleic acid construct further comprises a recombination target sequence. Optionally, the recombination target sequence is a FLP Recognition Target (“FRT”), lox, or Bxb1-recognized sequence. Optionally, the nucleotide sequence of interest is a first nucleotide sequence of interest, and the recombinant nucleic acid construct further comprises a second nucleotide sequence of interest.

Optionally, any of the above recombinant nucleic acid constructs may further comprise a GNMT gene.

In some embodiments provided herein comprising a nucleotide sequence of interest, the nucleotide sequence of interest encodes a polypeptide of interest or an RNA molecule of interest.

In some embodiments provided herein comprising a first nucleotide sequence of interest and a second nucleotide sequence of interest, the first nucleotide sequence of interest and the second nucleotide sequence of interest are transcribed as a single bicistronic mRNA transcript. Optionally, there is an IRES between the first nucleotide sequence of interest and second nucleotide sequence of interest. Optionally, the first nucleotide sequence of interest and second nucleotide sequence of interest are separately translated from the single bicistronic mRNA transcript into a first polypeptide and second polypeptide.

In some embodiments provided herein comprising a first nucleotide sequence of interest and a second nucleotide sequence of interest, the first nucleotide sequence of interest encodes a first polypeptide comprising an antibody variable light (VL) region and the second nucleotide sequence of interest encodes a second polypeptide comprising an antibody variable heavy (VH) region.

In some embodiments provided herein comprising a first nucleotide sequence of interest and a second nucleotide sequence of interest, the first nucleotide sequence of interest and the second nucleotide sequence of interest have the same nucleotide sequence (e.g. so that two copies of the nucleotide sequence of interest are included, for example, in a nucleic acid construct).

In some embodiments provided herein comprising a first nucleic acid construct and a second nucleic acid construct, the first nucleic acid construct and the second nucleic acid construct both contain at least a first nucleotide sequence of interest and a second nucleotide sequence of interest. For example, the first nucleotide sequence of interest may be a sequence which encodes a polypeptide comprising an antibody variable heavy (VH) region and the second nucleotide sequence of interest may be a sequence which encodes a polypeptide comprising an antibody variable light (VL) region. Thus, for example, a host cell containing the first nucleic acid construct and the second nucleic acid construct described above will contain at least two copies of the first nucleotide sequence of interest which encodes a polypeptide comprising an antibody variable heavy (VH) region and at least two copies of the second nucleotide sequence of interest which encodes a polypeptide comprising an antibody variable light (VL) region.

In some embodiments, a nucleic acid construct provided herein further comprises a gene encoding a recombinase or integrase for use with a recombination target sequence present on the nucleic acid construct.

In some embodiments, provided herein is a vector comprising a recombinant nucleic acid construct described herein. The vector may be, for example, a plasmid vector or a viral vector. The vector may further contain, for example, a selection marker such as an antibiotic selection marker, a glutamine synthetase selection marker, a hygromycin selection marker, a puromycin selection marker or a thymidine kinase selection marker.

In some embodiments, provided herein is a host cell containing one or more recombinant nucleic acid construct(s) or vector(s) provided herein. The recombinant nucleic acid construct(s) or vector(s) may be stably integrated into a chromosome of the host cell, or it may be episomal.

In some embodiments, a host cell may be a prokaryotic cell, a eukaryotic cell, a yeast cell, a plant cell, an animal cell, a mammalian cell, a mouse cell, a human cell, a CHO cell, a CHOK1 cell, or a CHOK1SV cell.

In some embodiments, also provided is the use of a host cell provided herein for the production of a polypeptide or RNA molecule encoded by a nucleotide sequence of interest.

In some embodiments, also provided is a recombinant polypeptide produced by a host cell provided herein.

In some embodiments, provided herein is a composition comprising A) a first recombinant nucleic acid construct comprising i) a first nucleotide sequence of interest and ii) a CBS gene and B) a second recombinant nucleic acid construct comprising i) a second nucleotide sequence of interest and ii) a CTH gene.

In some embodiments, also provided is a recombinant polypeptide provided herein and a pharmaceutically acceptable excipient.

In some embodiments, also provided is a host cell provided herein and a cell culture medium.

In some embodiments, also provided is a host cell provided herein, a recombinant nucleic acid construct provided herein, and a cell culture medium. Optionally, the host cell comprises a chromosome comprising a landing pad, wherein the landing pad comprises a recombination target site.

In some embodiments provided herein involving a cell culture medium, the medium is cysteine-deficient. Optionally, a cysteine-deficient medium provided herein comprises less than about 2 mM, less than about 1.8 mM, less than about 1.6 mM, less than about 1.5 mM, less than about 1.6 mM, less than about 1.4 mM, less than about 1.2 mM, less than about 1 mM, less than about 900 μM, less than about 800 μM, less than about 700 μM, less than about 600 μM, less than about 500 μM, less than about 100 μM, less than about 50 μM, less than about 10 μM, less than about 5 μM, less than about 1 μM, or 0 μM cysteine. Optionally, a cysteine-deficient medium comprises about 1 mM or less cysteine, 500 μM or less cysteine, 100 μM or less cysteine, 50 μM or less cysteine, 10 μM or less cysteine, 5 μM or less cysteine, 1 μM or less cysteine, or 0 μM cysteine.

In some embodiments, provided herein is a method of obtaining a host cell comprising an exogenous nucleotide sequence of interest, the method comprising: a) exposing a population of cells to an exogenous nucleic acid construct comprising the nucleotide sequence of interest, wherein the exogenous nucleic acid construct further comprises: i) a CBS gene, and ii) a CTH gene; b) culturing the population of cells exposed to the exogenous nucleic acid construct in a cysteine-deficient medium; and c) obtaining from the population of cells exposed to the exogenous nucleic acid construct a host cell comprising the exogenous nucleotide sequence of interest, wherein the host cell comprising the exogenous nucleotide sequence of interest comprises the exogenous nucleic acid construct, and wherein the host cell comprising the exogenous nucleotide sequence of interest has a greater ability to proliferate in a cysteine-deficient cell culture medium than a corresponding cell that does not contain the exogenous nucleic acid construct. Optionally, the exogenous nucleic acid construct further comprises a recombination target sequence. Optionally, a chromosome of the host cell comprises a first landing pad, wherein the first landing pad comprises a recombination target site. Optionally, the nucleic acid construct recombination target sequence and the chromosomal recombination target site are FLP, lox, or Bxb1 sequences.

In some embodiments, provided herein is a method of obtaining a cell comprising a first exogenous nucleotide sequence of interest and a second exogenous nucleotide sequence of interest, the method comprising: a) exposing a population of cells to I) a first exogenous nucleic acid construct comprising i) the first exogenous nucleotide sequence of interest and ii) a CBS gene, and II) a second exogenous nucleic acid construct comprising i) the second exogenous nucleotide sequence of interest and ii) a CTH gene; and b) culturing the population of cells exposed to the first exogenous nucleic acid construct and the second exogenous nucleic acid construct in a cysteine-deficient medium; and c) obtaining from the population of cells exposed to the first exogenous nucleic acid construct and the second exogenous nucleic acid construct a host cell comprising the first exogenous nucleotide sequence of interest and the second exogenous nucleotide sequence of interest, wherein the host cell comprising the first exogenous nucleotide sequence of interest and the second exogenous nucleotide sequence of interest comprises the first exogenous nucleic acid construct and the second exogenous nucleic acid construct, and wherein the host cell comprising the first exogenous nucleic acid construct and the second exogenous nucleic acid construct has a greater ability to proliferate in a cysteine-deficient cell culture medium than a corresponding cell that does not contain the first exogenous nucleic acid construct and the second exogenous nucleic acid. Optionally, the first exogenous nucleic acid construct further comprises a recombination target sequence. Optionally, the second exogenous nucleic acid construct further comprises a recombination target sequence. Optionally, the first exogenous nucleic acid construct further comprises a first recombination target sequence, and the second exogenous nucleic acid construct further comprises a second recombination target sequence. Optionally, a chromosome of the host cell comprises a first landing pad and a second landing pad, wherein the first landing pad comprises a first recombination target site and the second landing pad comprises a second recombination target site. Optionally, a first chromosome of the host cell comprises a first landing pad, wherein the first landing pad comprises a first recombination target site, and a second chromosome of the host cell comprises a second landing pad, wherein the second landing pad comprises a second recombination target site. Optionally, the nucleic acid construct recombination target sequences and the chromosomal recombination target sites comprise FLP, lox, or Bxb1 sequences.

In some embodiments, provided herein is a method of producing a host cell comprising an exogenous nucleotide sequence of interest, the method comprising: a) introducing into a host cell an exogenous nucleic acid construct comprising the nucleotide sequence of interest, wherein the exogenous nucleic acid construct further comprises: i) a CBS gene, and ii) a CTH gene; b) culturing the host cell comprising the exogenous nucleic acid construct in a cysteine-deficient medium, wherein the host cell comprising the exogenous nucleic acid construct proliferates more rapidly in the cysteine-deficient medium than a corresponding otherwise identical host cell that lacks the exogenous nucleic acid construct. Optionally, the exogenous nucleic acid construct is stably integrated into a chromosome of the host cell. Optionally, the exogenous nucleic acid construct is stably integrated into the chromosome by homologous recombination between the exogenous nucleic acid construct and the chromosome. Optionally, the integration of the exogenous nucleic acid construct into the chromosome is facilitated by a viral vector or an exogenous nuclease.

In some embodiments, provided herein is a method of producing a host cell comprising a first exogenous nucleotide sequence of interest and a second exogenous nucleotide sequence of interest, the method comprising: a) introducing into a host cell I) a first exogenous nucleic acid construct comprising i) the first exogenous nucleotide sequence of interest and ii) a CBS gene and II) a second exogenous nucleic acid construct comprising i) the second exogenous nucleotide sequence of interest and ii) a CTH gene; and b) culturing the host cell comprising the first exogenous nucleic acid construct and the second exogenous nucleic acid construct in a cysteine-deficient medium, wherein the host cell comprising the first exogenous nucleic acid construct and the second exogenous nucleic acid construct proliferates more rapidly in the cysteine-deficient medium than a corresponding otherwise identical host cell that lacks the first exogenous nucleic acid construct and second exogenous nucleic acid construct. Optionally, the first exogenous nucleic acid construct and the second exogenous nucleic acid construct are both stably integrated into a first chromosome of the host cell, or the first exogenous nucleic acid construct is stably integrated into a first chromosome of the host cell and the second exogenous nucleic acid construct is stably integrated into a second chromosome of the host cell. Optionally, the first exogenous nucleic acid construct and the second exogenous nucleic acid construct are stably integrated into the chromosome by homologous recombination between the respective exogenous nucleic acid construct and the chromosome. Optionally, the integration of the exogenous nucleic acid constructs is facilitated by a viral vector or an exogenous nuclease. Optionally, the viral vector is an adeno-associated virus vector that mediates homologous recombination.

In some embodiments, provided herein is a host cell comprising an exogenous copy of a CBS gene and a CTH gene. Optionally, the exogenous CBS gene and CTH gene are in a plasmid in the cell. Optionally, the exogenous CBS gene and CTH gene are stably integrated into a first chromosomal locus and a second chromosomal locus in the cell, respectively. Optionally, the exogenous CBS gene and the exogenous CTH are both operably linked to a promoter. Optionally the host cell comprising an exogenous copy of the CBS gene and CTH gene has a greater ability to proliferate in a cysteine-deficient media that a corresponding host cell that does not contain the exogenous CBS gene and CTH gene. In some embodiments, also provided herein is a method of a making a host cell provided above. Optionally, the method comprises introducing one or more nucleic acid constructs comprising the exogenous CBS gene and the CTH gene into the host cell. Optionally, the exogenous CBS gene and CTH gene are operably linked in the nucleic acid construct to a promoter sequence.

In some embodiments, provided herein is a host cell which has been genetically modified such to have increased gene expression of the endogenous CBS gene and endogenous CTH gene in the cell. Optionally, such a host cell may be modified by, for example, genetically modifying a promoter or enhancer sequence operably linked to the CBS or CTH gene to increase the expression of the respective gene, or by inserting an exogenous promoter or enhancer sequence into a chromosomal locus such that it is operably linked to the endogenous CBS or endogenous CTH gene, and such that the cell has increased gene expression of the respective genes. Optionally, the host cell has a greater ability to proliferate in a cysteine-deficient media that a corresponding host cell that does not have increased expression of the CBS gene and CTH gene. In some embodiments, also provided herein is a method of a making a host cell provided above. Optionally, the method comprises introducing one or more nucleic acid constructs comprising promoter sequences into the host cell. Optionally, the nucleic acid construct(s) are integrated into one or more chromosomes of the host cell, such that expression of the endogenous CBS gene and the endogenous CTH gene is increased.

In some embodiments provided herein, a CBS gene encodes a polypeptide comprising the amino acid sequence shown in SEQ ID NO: 1, or a sequence with at least 99%, 98%, 97%, 96%, 95%, 90%, 85%, 80%, 75%, 70%, 65%, 60%, 55%, or 50% homology thereof. In some embodiments, a CBS polypeptide comprises the amino acid sequence shown in SEQ ID NO: 1, or a sequence with at least 99%, 98%, 97%, 96%, 95%, 90%, 85%, 80%, 75%, 70%, 65%, 60%, 55%, or 50% homology thereof.

In some embodiments provided herein, a CBS gene comprises a DNA sequence shown in SEQ ID NO: 2, or a sequence with at least 99%, 98%, 97%, 96%, 95%, 90%, 85%, 80%, 75%, 70%, 65%, 60%, 55%, or 50% homology thereof.

In some embodiments provided herein, a CTH gene encodes a polypeptide comprising the amino acid sequence shown in SEQ ID NO: 3, or a sequence with at least 99%, 98%, 97%, 96%, 95%, 90%, 85%, 80%, 75%, 70%, 65%, 60%, 55%, or 50% homology thereof. In some embodiments, a CTH polypeptide comprises the amino acid sequence shown in SEQ ID NO: 3, or a sequence with at least 99%, 98%, 97%, 96%, 95%, 90%, 85%, 80%, 75%, 70%, 65%, 60%, 55%, or 50% homology thereof.

In some embodiments provided herein, a CTH gene comprises a DNA sequence shown in SEQ ID NO: 4, or a sequence with at least 99%, 98%, 97%, 96%, 95%, 90%, 85%, 80%, 75%, 70%, 65%, 60%, 55%, or 50% homology thereof.

In some embodiments provided herein, a GNMT gene comprises a DNA sequence as shown in GenBank Accession BC014283, or sequence with at least 99%, 98%, 97%, 96%, 95%, 90%, 85%, 80%, 75%, 70%, 65%, 60%, 55%, or 50% homology thereof. Corresponding GNMT polypeptides are also provided.

### BRIEF DESCRIPTION OF THE FIGS./DRAWINGS

FIG. 1 depicts a graph summarizing recovery profiles of cells transfected with vectors containing CBS and/or CTH genes or corresponding control vectors and selected for antibiotic resistance.

FIG. 2 depicts a schematic outlining an exemplary strategy for selecting a pool of cells transfected with vectors containing exogenous CBS and CTH genes, wherein the cells are selected for cysteine prototrophy.

FIG. 3 depicts transfection recovery, under antibiotic selection, of CTH+CBS cell pools that were transfected with the GNMT transgene (solid circle) and the CTH+CBS cell pools transfected with a control vector (“GNMT Ctrl”) containing the gene chloramphenicol acetyltransferase (CAT) (solid triangle). The X-axis shows days post-transfection and the Y-axis shows % cell viability.

FIG. 4 depicts viability over time of cells overexpressing CBS, CTH, and GNMT (solid circle) or CTH and CBS (solid square) in media without L-cysteine and with L-homocysteine (solid line) or without L-homocysteine (dotted line). The X-axis shows time (days) and the Y-axis shows % cell viability.

FIG. 5 shows adaptation of CBS, CTH, and GNMT overexpressing cells (solid circle) and CTH and CBS overexpressing cells (solid square) from media that lacks L-cysteine to media that lacks both L-cysteine and L-homocysteine. Upper panel: Concentration of L-homocysteine (mM) as indicated; X-axis: time (days); Y-axis: viable cell density; Lower panel: X-axis: time (days); Y-axis: % cell viability.

FIG. 6 shows growth characteristics of CBS, CTH, and GNMT overexpressing cells (solid circle) and CTH and CBS overexpressing cells (solid square) in L-Cysteine and L-Homocysteine free media in a Fed-Batch process. The X-axis show time (days) and the Y-axis shows viable cell density.

FIG. 7A shows impact of MTR inhibition by sodium nitroprusside (SNP) in CBS, CTH, and GNMT overexpressing cells in L-Cysteine and L-Homocysteine free media. In the upper panel, the X-axis show time (days) and the Y-axis shows viable cell density. In the lower panel, the X-axis show time (days) and the Y-axis shows percent viability. In both panels, the different media conditions are notated as: empty circle: CD CHO media without L-cysteine or L-homocysteine (positive control); empty diamond: Pfizer internal medium with vitamin B12 (IMB12) without L-cysteine or L-homocysteine (negative control); empty triangle: IMB12 with 10 micromolar (uM) sodium nitroprusside; empty square: IMB12 with 1 uM sodium nitroprusside; solid circle: IMB12 with 0.5 uM sodium nitroprusside; solid diamond: IMB12 with 0.25 uM sodium nitroprusside.

FIG. 7B shows impact of MTR inhibition by sodium nitroprusside (SNP) in CBS and CTH overexpressing cells in L-Cysteine and L-Homocysteine free media. In the upper panel, the X-axis show time (days) and the Y-axis shows viable cell density. In the lower panel, the X-axis show time (days) and the Y-axis shows percent viability. In both panels, the different media conditions are notated as: empty circle: CD CHO media without L-cysteine or L-homocysteine (positive control); empty diamond: Pfizer internal medium with vitamin B12 (IMB12) without L-cysteine or L-homocysteine (negative control); empty triangle: IMB12 with 10 micromolar (uM) sodium nitroprusside; empty square: IMB12 with 1 uM sodium nitroprusside; solid circle: IMB12 with 0.5 uM sodium nitroprusside; solid diamond: IMB12 with 0.25 uM sodium nitroprusside.

FIG. 8 shows adaptation of CBS, CTH, and GNMT overexpressing cells (“GNMT”; solid and empty circles) and CTH and CBS overexpressing cells (“CTH+CBS”; solid and empty squares) to media that lacks beta-mercaptoethanol (BME), L-cysteine, and L-homocysteine. The cells were previously adapted to grow in media that lacks L-cysteine, and L-homocysteine but that contained 50 uM BME. Upper panel: X-axis: time (days); Y-axis: viable cell density; Lower panel: X-axis: time (days); Y-axis: % cell viability. In both panels, the different conditions are notated as: empty circle: GNMT cells with BME; solid circle: GNMT cells without BME; empty square: CTH+CBS cells with BME; solid square: CTH+CBS cells without BME.

FIG. 9 shows average doubling times of 6 GNMT clones (overexpressing CTH, CBS, and GNMT; clones 3, 10, 12, 15, 16, and 19), CTH+CBS cell pools (overexpressing CTH and CBS) and a transfection control (wild type cells that underwent transfection protocol without DNA). The GNMT clones were grown in BME-free, L-Cysteine/L-Homocysteine free media. The CTH+CBS cell pools were grown in L-Cysteine/L-Homocysteine free media with or without BME as indicated in the chart. The transfection control was grown in BME-free L-Cysteine containing media. X-axis: different clones or conditions as indicated; Y-axis: hours.

FIG. 10A shows growth characteristics and titer of GNMT clones (overexpressing CTH, CBS, and GNMT; clones 3, 10, 12, 15, 16, and 19; empty triangle, solid circle, horizontal line, solid square, empty square, and empty triangle, respectively) and a CBS+CTH cell pool (empty circle) in a fed-batch process. The GNMT clones were grown in BME-free, L-cysteine/L-homocysteine free environment. The CTH+CBS cell pools were grown in L-cysteine/L-homocysteine free environment with BME. Upper panel: X-axis: time (days); Y-axis: viable cell density; Lower panel: X-axis: time (days); Y-axis: % cell viability. In both panels, the different conditions are notated as: GNMT clones 3, 10, 12, 15, 16, and 19: empty triangle, solid circle, horizontal line, solid square, empty square, and empty triangle, respectively; CBS+CTH cell pool: empty circle.

FIG. 10B shows harvest titer levels of IgG antibody on Day 10 of the 6 GNMT clones and the CBS+CTH pool of FIG. 10A. X-axis: different clones or conditions as indicated; Y-axis: day 10 titer (mg/L).

FIG. 11A shows impact of supplementing cell culture with Oleic Acid (OA) or Ferrostatin1 (Fer1) on growth characteristics of two GNMT clones (overexpressing CTH, CBS, and GNMT; clones 15 and 19) in L-Cysteine/L-Homocysteine free media. Upper panel: X-axis: time (days); Y-axis: viable cell density; Lower panel: X-axis: time (days); Y-axis: % cell viability. In both panels, the different conditions are notated as: clone 15 control conditions (no OA or Fer1): empty square; clone 15 Fer1 supplementation: empty triangle; clone 15 OA supplementation: empty circle; clone 19 control conditions (no OA or Fer1): solid square; clone 19 Fer1 supplementation: solid triangle; clone 19 OA supplementation: solid circle.

FIG. 11B shows impact of supplementing cell culture with vitamin K1 (VitK1) on growth characteristics of two GNMT clones (overexpressing CTH, CBS, and GNMT; clones 15 and 19) in L-Cysteine/L-Homocysteine free media. Upper panel: X-axis: time (days); Y-axis: viable cell density; Lower panel: X-axis: time (days); Y-axis: % cell viability. In both panels, the different conditions are notated as: clone 15 control conditions (no VitK1): solid circle; clone 15 VitK1 supplementation: empty circle; clone 19 control conditions (no VitK1): solid triangle; clone 19 VitK1 supplementation: empty triangle.

### DETAILED DESCRIPTION

Disclosed here are compositions and methods for conferring cysteine prototrophy on cells, uses for these compositions and methods, and related methods and materials such as nucleic acid constructs, cells, and cell culture medium.

The invention provided herein relates to compositions and methods wherein cells which are cysteine auxotrophs (i.e. which cannot synthesize sufficient quantities of cysteine for normal growth, and which must be provided with a medium that contains cysteine) are converted to cysteine prototrophs (i.e. which can synthesize sufficient quantities of cysteine for normal growth, and which can grow in cysteine-deficient media) by the introduction of exogenous CBS and CTH genes into the cell, such that expression of the CBS and CTH genes in the cell is increased. Optionally, an exogenous GNMT gene may also be introduced into the cell, such that expression of the GNMT gene in the cell is increased. (Commonly, a host cell into which the CBS, CTH, and optionally GNMT genes are introduced according to methods provided herein already contains endogenous CBS, CTH, GNMT genes; however these endogenous genes are not expressed or are only expressed at a low level.) Increased expression of the CBS, CTH, and optionally GNMT genes and the resulting increased enzymatic activity of the CBS, CTH, and optionally GNMT polypeptides in such cells permits the cells to grow in cysteine-deficient media, and thus, host cells that are transfected with recombinant copies of the CBS, CTH, and optionally GNMT genes can be selected. Accordingly, in one aspect, provided herein is a cysteine selectable marker system. The cysteine selectable marker system comprises one or more recombinant nucleic acid constructs containing the CBS, CTH, and optionally GNMT genes, and methods of using the constructs.

In another aspect, cells containing increased expression of the CBS, CTH, and optionally GNMT genes may be selected by any method known in the art (e.g. antibiotic selection or selection based on growth characteristics), and these cells may be used for recombinant protein production or other cell-based production processes. Such cells have greater ability to proliferate in cysteine-deficient media as compared to otherwise identical cells that do not have increased expression of CBS, CTH, and optionally GNMT.

Further provided herein are various applications relating to the cysteine selectable marker system. For example, provided herein are compositions and methods for selecting a host cell that contains an exogenous nucleotide sequence of interest, in which the nucleotide sequence of interest is coupled in a recombinant nucleic acid construct to one or both of the CBS and CTH genes, and optionally the GNMT gene. In compositions and methods provided herein, the CBS, CTH, and optionally GNMT genes may be provided together in a single nucleic acid construct, or they may be provided in separate nucleic acid constructs. For some purposes, it may be beneficial to provide the CBS, CTH, and optionally GNMT genes together in a single nucleic acid construct (e.g. in situations in which it is desirable to introduce only a single exogenous nucleic acid construct into a host cell); alternatively, for some purposes, it may be beneficial to provide the CBS, CTH, and optionally GNMT genes in separate nucleic acid constructs (e.g. in situations in which it is desirable to introduce two separate exogenous nucleic acid constructs into a host cell).

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995), as well as in subsequent editions and corresponding websites of the above references, as applicable.

### Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA₁ and IgA₂. The heavy-chain constant regions that correspond to the different classes of immunoglobulins are called alpha, delta, epsilon, gamma, and mu, respectively. The subunit structures and three-dimensional configurations of different classes of immunoglobulins are well known.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example. As used herein, “humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As known in the art, “polynucleotide,” or “nucleic acid,” as used interchangeably herein, refer to chains of nucleotides of any length and conformation (e.g. linear or circular) and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” or “genetic control element”, used interchangeably herein, means a nucleic acid sequence that regulates transcription of a nucleic acid. An expression control sequence can be a promoter, such as a constitutive or an inducible promoter, or an enhancer. The expression control sequence is operably linked to the nucleic acid sequence to be transcribed.

As used herein, a “recombinant” nucleic acid refers to a nucleic acid molecule that contains a polynucleotide sequence that does not occur in nature and/or or which is synthetically manufactured. For example, a “recombinant” nucleic acid may contain a protein encoding gene coupled to a vector sequence. The sequence of the protein encoding gene may occur in nature, but the gene does not naturally occur in combination with the vector sequence. Put another way, a “recombinant” nucleic acid molecule may contain as part of the molecule a nucleic acid sequence that occurs in nature, but that sequence is either coupled to another sequence (such that the totality of the nucleic acid molecule sequence does not occur in nature) and/or the molecule is synthetically manufactured. A “recombinant” polypeptide refers to a polypeptide produced from a recombinant nucleic acid.

As used herein, an “exogenous” nucleic acid molecule refers to a recombinant nucleic acid molecule that will be or has been introduced into a host cell (e. g. by conventional genetic engineering methods, preferably by means of transformation, electroporation, lipofection, or transfection), which was prior to said introduction was not present in said host cell. Such sequences are also termed “transgenic”. An exogenous nucleic acid molecule may contain a nucleotide sequence of that is the same as a sequence that is endogenous to the cell (i.e. an exogenous nucleic acid molecule may contain a nucleotide sequence of a gene that is endogenous to the host cell, such that introduction of the exogenous nucleic acid molecule into the host cell introduces a second copy of the gene into the host cell). References herein to an “exogenous” gene refer to an exogenous nucleic acid containing a nucleotide sequence encoding the referenced gene.

As used herein, the term “site” refers to a nucleotide sequence, in particular a defined stretch of nucleotides, i. e. a defined length of a nucleotide sequence, preferably a defined stretch of nucleotides being part of a larger stretch of nucleotides. In some embodiments, a site, e. g. a site which is a “hot-spot”, is part of a genome. In some embodiments, a site is introduced into a genome, e. g. a recombination target site.

References herein to a “first chromosome” and “second chromosome” or the like are to be understood to refer to the relationship between the two chromosomes (or other respective object), rather than any particular chromosome of the cell. Thus, for example, when a “first chromosome” and “second chromosome” are mentioned in a common sentence or description, these terms simply indicate that the referenced chromosomes are different from each other; they do not refer to any specific chromosome of the cell.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, PA, 1990; and Remington, The Science and Practice of Pharmacy 20th Ed. Mack Publishing, 2000).

“Cysteine” refers to the amino acid encoded by the codons UGU and UGC. Cysteine has the IUPAC name “cysteine” and has the CAS number 52-90-4, and chemical formula C₃H₇NO₂S. References to “cysteine” herein refer to L-cysteine, unless otherwise noted.

“Homocysteine” is a non-proteinogenic amino acid. Homocysteine has the IUPAC name 2-amino-4-sulfanylbutanoic acid, and the CAS number 454-29-5 (racemate) and 6027-13-0 (L-isomer). Homocysteine has the chemical formula C₄H₉NO₂S References to “homocysteine” herein refer to L-homocysteine, unless otherwise noted.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting. The term “or” when used in the context of a listing of multiple options (e.g. “A, B, or C”) shall be interpreted to include any one or more of the options, unless the context clearly dictates otherwise.

Exemplary methods and materials are described herein, although methods and materials similar or equivalent to those described herein can also be used in the practice or testing of the present invention. The materials, methods, and examples are illustrative only and not intended to be limiting.

### Cysteine Selectable Marker System

In some embodiments, provided herein is a cysteine selectable marker system. The cysteine selectable marker system includes, for example, recombinant nucleic acid constructs and vectors that contain the CBS and/or CTH genes as described herein, and uses thereof. The cysteine selectable marker system involves the introduction of the CBS and CTH genes into a host cell; accordingly selection of a host cell containing one or more exogenous nucleic acids via the use of the cysteine selectable marker system as provided herein involves a host cell receiving both the CBS and CTH genes. These genes may be introduced into a host cell on the same nucleic acid construct, or they may be provided on separate nucleic acid constructs. Nucleotide sequences of interest may be coupled to the CBS and CTH genes in one or more nucleic acid constructs, and the cells transfected with a construct or constructs containing the nucleotide sequences of interest may thus be selected for via selection of cells that contain the CBS and CTH genes; such cells may in turn be selected via selection of cells which exhibit cysteine prototrophy.

### Nucleic Acid Constructs and Vectors

### CBS Gene

Embodiments provided herein may include a CBS gene. The CBS gene encodes the enzyme cystathionine beta-synthase (“CBS”). CBS catalyzes the conversion of homocysteine to cystathionine. Exemplary CBS gene and polypeptide sequences are provided via GenBank Accession Nos. BC013472 (mouse) and BC011381 (human).

An exemplary CBS polypeptide is, for example, the mouse CBS amino acid sequence shown in SEQ ID NO: 1 (MPSGTSQCEDGSAGGFQHLDMHSEKRQLEKGPSGDKDRVWIRPDTPSRCTWQLG RAMADSPHYHTVLTKSPKILPDILRKIGNTPMVRINKISKNAGLKCELLAKCEFFNAGGS VKDRISLRMIEDAERAGNLKPGDTIIEPTSGNTGIGLALAAAVKGYRCIIVMPEKMSMEK VDVLRALGAEIVRTPTNARFDSPESHVGVAWRLKNEIPNSHILDQYRNASNPLAHYDD TAEEILQQCDGKLDMLVASAGTGGTITGIARKLKEKCPGCKIIGVDPEGSILAEPEELNQ TEQTAYEVEGIGYDFIPTVLDRAVVDKWFKSNDEDSFAFARMLIAQEGLLCGGSSGSA MAVAVKAARELQEGQRCVVILPDSVRNYMSKFLSDKWMLQKGFMKEELSVKRPWW WRLRVQELSLSAPLTVLPTVTCEDTIAILREKGFDQAPVVNESGAILGMVTLGNMLSSL LAGKVRPSDEVCKVLYKQFKPIHLTDTLGTLSHILEMDHFALVVHEQIQYCSNGMSSKQ QMVFGVVTAIDLLNFVAAREQTQT). In some embodiments, a CBS polypeptide is a polypeptide that has at least 99%, 98%, 97%, 96%, 95%, 90%, 85%, 80%, 75%, 70%, 65%, 60%, 55%, or 50% homology with the amino acid sequence shown in SEQ ID NO: 1. In some embodiments, a CBS polypeptide is a catalytically active fragment of any of the CBS polypeptides described above.

An exemplary CBS gene sequence is, for example, the mouse CBS cDNA sequence shown in SEQ ID NO: 2 (ATGCCTTCAGGGACATCCCAGTGTGAAGATGGCTCTGCTGGGGGCTTCCAGCAC TTGGACATGCACTCAGAAAAGAGACAACTGGAGAAGGGCCCCTCAGGGGACAAG GATCGAGTCTGGATCCGGCCTGATACCCCAAGCAGATGTACCTGGCAGCTGGGC AGGGCCATGGCGGACTCCCCACATTATCACACAGTGCTGACCAAATCCCCCAAAA TTTTACCAGATATTCTGAGGAAAATTGGGAACACCCCTATGGTCAGAATCAACAAG ATCTCAAAGAATGCCGGTCTCAAGTGTGAGCTCTTGGCCAAGTGTGAGTTCTTCAA TGCGGGTGGGAGTGTGAAGGACCGCATCAGCCTTCGGATGATCGAAGATGCTGA GCGAGCTGGAAACTTGAAGCCTGGAGACACTATCATTGAGCCAACTTCTGGCAAC ACAGGGATCGGGCTGGCTCTGGCTGCTGCAGTGAAGGGCTATCGCTGCATTATC GTGATGCCGGAGAAGATGAGTATGGAGAAGGTGGATGTGCTGCGGGCTCTGGGA GCCGAGATTGTGAGGACGCCCACCAATGCCAGATTTGATTCCCCCGAGTCCCACG TGGGAGTGGCATGGCGACTGAAGAACGAAATCCCTAATTCTCACATTCTGGACCA GTACCGCAATGCCAGCAACCCTTTGGCACACTACGATGACACCGCCGAGGAGATC CTGCAGCAGTGTGACGGGAAGCTGGATATGCTGGTGGCTTCAGCAGGCACGGGT GGCACCATCACAGGGATCGCCAGAAAGCTGAAGGAGAAGTGCCCTGGCTGTAAA ATCATCGGTGTCGATCCTGAAGGCTCCATCCTTGCGGAGCCCGAGGAGCTGAACC AGACGGAGCAAACAGCCTATGAGGTGGAAGGGATTGGCTACGACTTCATCCCGAC AGTCCTGGACAGGGCGGTGGTGGATAAGTGGTTCAAGAGCAACGATGAAGATTCC TTCGCCTTTGCCCGCATGCTCATCGCACAGGAAGGACTGCTATGTGGTGGAAGCT CTGGCAGCGCCATGGCTGTGGCTGTGAAGGCTGCCCGGGAGCTGCAGGAAGGG CAGCGCTGTGTGGTCATCCTGCCTGACTCTGTGCGGAACTACATGTCCAAGTTCC TGAGTGACAAATGGATGCTGCAGAAAGGTTTCATGAAAGAGGAGCTCTCAGTGAA GAGGCCCTGGTGGTGGCGTCTGCGTGTTCAAGAGCTGAGCCTGTCGGCCCCGCT GACCGTGTTGCCCACGGTCACCTGTGAGGACACCATCGCCATCCTCCGGGAGAA GGGTTTTGACCAGGCACCTGTGGTCAACGAGTCTGGGGCCATCCTAGGGATGGT GACCCTCGGGAACATGCTGTCATCCCTGCTGGCTGGAAAGGTGCGGCCATCAGA CGAAGTCTGCAAAGTCCTCTACAAGCAGTTCAAACCGATCCACCTGACCGACACG CTGGGCACACTCTCTCACATCCTGGAGATGGACCACTTCGCCCTGGTGGTCCACG AGCAGATCCAATACTGCAGCAATGGCATGTCCAGCAAGCAGCAGATGGTGTTTGG GGTTGTCACTGCCATTGACCTGCTAAACTTCGTGGCAGCCCGTGAGCAGACCCAG ACATAG). In some embodiments, a CBS gene sequence is a nucleotide sequence that has at least 99%, 98%, 97%, 96%, 95%, 90%, 85%, 80%, 75%, 70%, 65%, 60%, 55%, or 50% homology with the nucleotide sequence shown in SEQ ID NO: 2. In some embodiments, a CBS gene encodes a catalytically-active fragment of any of the CBS polypeptides described above.

### CTH Gene

Embodiments provided herein may include a CTH gene. The CTH gene encodes the enzyme cystathionase (cystathionine gamma-lyase) (“CTH”) (also known as “CSE”. CTH catalyzes the conversion of cystathionine into cysteine, alpha-ketobutyrate, and ammonia. Exemplary CTH gene and polypeptide sequences are provided via GenBank Accession Nos. BC019483 (mouse), BC015807 (human), and BC078869 (rat).

An exemplary CTH polypeptide is, for example, the mouse CTH amino acid sequence shown in SEQ ID NO: 3 (MQKDASLSGFLPSFQHFATQAIHVGQEPEQWNSRAVVLPISLATTFKQDFPGQSSGF EYSRSGNPTRNCLEKAVAALDGAKHSLAFASGLAATITITHLLKAGDEIICMDEVYGGT NRYFRRVASEFGLKISFVDCSKTKLLEAAITPQTKLVWIETPTNPTLKLADIGACAQIVHK RGDIILVVDNTFMSAYFQRPLALGADICMCSATKYMNGHSDVVMGLVSVNSDDLNSRL RFLQNSLGAVPSPFDCYLCCRGLKTLQVRMEKHFKNGMAVARFLETNPRVEKVVYPG LPSHPQHELAKRQCSGCPGMVSFYIKGALQHAKAFLKNLKLFTLAESLGGYESLAELP AIMTHASVPEKDRATLGINDTLIRLSVGLEDEQDLLEDLDRALKAAHP). In some embodiments, a CTH polypeptide is a polypeptide that has at least 99%, 98%, 97%, 96%, 95%, 90%, 85%, 80%, 75%, 70%, 65%, 60%, 55%, or 50% homology with the amino acid sequence shown in SEQ ID NO: 3. In some embodiments, a CTH polypeptide is a catalytically active fragment of any of the CTH polypeptides described above.

An exemplary CTH gene sequence is, for example, the mouse CTH cDNA sequence shown in SEQ ID NO: 4 (ATGCAGAAGGACGCCTCTTTGAGCGGCTTCCTGCCTAGTTTCCAGCATTTCGCCA CTCAGGCCATCCACGTGGGACAAGAGCCTGAGCAATGGAATTCTCGTGCCGTGGT GCTGCCCATTTCGTTGGCCACCACATTTAAGCAGGACTTCCCGGGCCAGTCCTCG GGTTTTGAATACAGCCGCTCTGGAAATCCAACAAGGAATTGCTTGGAAAAAGCAGT GGCTGCGTTGGATGGGGCAAAGCACAGTTTGGCCTTTGCATCGGGTCTTGCTGCC ACCATTACGATTACCCATCTTTTAAAAGCAGGAGATGAAATCATTTGCATGGATGAA GTGTATGGAGGCACCAACAGGTACTTCAGGAGGGTGGCATCTGAATTTGGACTGA AGATTTCTTTTGTAGATTGTTCCAAAACCAAATTGCTAGAGGCAGCGATTACACCAC AAACCAAGCTTGTTTGGATCGAAACACCCACAAACCCAACTTTGAAGTTGGCTGAC ATTGGAGCCTGCGCACAAATTGTCCACAAACGTGGAGACATCATTTTGGTTGTAGA TAACACCTTCATGTCTGCATATTTCCAGAGACCTTTGGCTCTGGGTGCTGATATTT GTATGTGTTCTGCCACAAAATACATGAATGGCCACAGCGATGTTGTCATGGGTTTA GTGTCTGTTAATTCTGATGACCTCAATAGTCGGCTTCGTTTCCTGCAGAATTCACTA GGAGCAGTTCCTTCTCCTTTTGATTGTTACCTCTGCTGCCGAGGCCTGAAGACACT GCAGGTCCGGATGGAGAAACATTTCAAGAATGGGATGGCGGTGGCTCGTTTCCTG GAGACCAATCCCCGGGTAGAAAAGGTTGTTTATCCTGGGCTACCCTCTCACCCTC AGCATGAGCTGGCCAAACGCCAGTGCTCGGGCTGCCCAGGGATGGTCAGTTTCT ACATCAAGGGTGCTCTGCAGCATGCTAAGGCCTTCCTCAAAAATCTAAAGCTGTTT ACTCTGGCAGAGAGCCTGGGAGGATATGAGAGTCTGGCTGAGCTTCCAGCAATCA TGACCCATGCCTCTGTGCCTGAGAAGGACAGAGCTACCCTCGGGATCAATGACAC ACTGATACGACTTTCTGTGGGCCTAGAGGATGAACAGGACCTTCTTGAAGACCTG GATCGAGCTTTGAAGGCAGCGCACCCTTAA). In some embodiments, a CTH gene sequence is a nucleotide sequence that has at least 99%, 98%, 97%, 96%, 95%, 90%, 85%, 80%, 75%, 70%, 65%, 60%, 55%, or 50% homology with the nucleotide sequence shown in SEQ ID NO: 4. In some embodiments, a CTH gene encodes a catalytically-active fragment of any of the CTH polypeptides described above.

### GNMT Gene

Embodiments provided herein may include a GNMT gene. The GNMT gene encodes the enzyme glycine N-methyltransferase (“GNMT”). GNMT catalyzes the conversion of S-adenosyl-L-methionine+glycine to S-adenosyl-L-homocysteine+sarcosine. Exemplary GNMT gene and polypeptide sequences are provided via GenBank Accession Nos. BC014283 (mouse) and 27232 (human).

An exemplary GNMT polypeptide is, for example, the mouse GNMT amino acid sequence shown in SEQ ID NO: 5 (MVDSVYRTRSLGVAAEGLPDQYADGEAARVWQLYIGDTRSRTAEYKAWLLGLLRQH GCHRVLDVACGTGVDSIMLVEEGFSVMSVDASDKMLKYALKERWNRRKEPSFDNWV IEEANWLTLDKDVLSGDGFDAVICLGNSFAHLPDCKGDQSEHRLELKNIASMVRPGGL LVIDHRNYDYILSTGCAPPGKNIYYKSDLTKDITTSVLTVNNKAHMVTLDYTVQVPGTG RDGSPGFSKFRLSYYPHCLASFTELVRAAFGGRCQHSVLGDFKPYKPGQAYVPCYFI HVLKKTD). In some embodiments, a GNMT polypeptide is a polypeptide that has at least 99%, 98%, 97%, 96%, 95%, 90%, 85%, 80%, 75%, 70%, 65%, 60%, 55%, or 50% homology with the amino acid sequence shown in SEQ ID NO: 5. In some embodiments, a GNMT polypeptide is a catalytically active fragment of any of the GNMT polypeptides described above.

An exemplary GNMT gene sequence is, for example, the mouse GNMT cDNA sequence shown in SEQ ID NO: 6

(CCACGCGTCCGGGATGGTGGACAGCGTGTACCGTACCCGCTCCCTGGGG
GTGGCGGCCGAAGGGCTCCCGGACCAGTATGCAGATGGGGAGGCCGCACG
TGTGTGGCAGCTGTACATCGGGGACACCCGCAGCCGTACCGCAGAGTACA
AGGCGTGGTTGCTTGGGCTGTTGCGCCAGCACGGGTGCCACAGGGTGCTG
GACGTAGCCTGTGGCACAGGAGTGGACTCCATCATGCTGGTGGAAGAGGG
CTTCAGCGTGATGAGCGTGGACGCCAGCGACAAGATGCTGAAATATGCGC
TTAAGGAGCGCTGGAACCGGAGGAAAGAGCCATCCTTTGACAATTGGGTC
ATTGAAGAAGCCAACTGGTTGACGCTGGACAAAGATGTGCTTTCAGGAGA
TGGCTTTGATGCTGTCATCTGCCTTGGGAACAGTTTTGCTCACTTGCCAG
ACTGCAAAGGTGACCAGAGCGAGCACCGGCTGGAACTAAAGAACATTGCA
AGCATGGTGCGGCCCGGGGGCCTGCTGGTGATCGACCACCGCAACTACGA
CTATATCCTCAGCACAGGCTGTGCGCCCCCGGGGAAGAACATCTACTATA
AGAGTGACCTGACCAAGGACATTACGACGTCAGTACTGACAGTCAACAAC
AAAGCCCACATGGTAACCCTGGACTACACAGTGCAGGTGCCAGGCACTGG
CAGAGATGGCTCTCCTGGCTTCAGTAAGTTCCGGCTCTCTTACTACCCAC
ACTGTTTGGCGTCTTTCACGGAGTTGGTGCGAGCAGCCTTTGGGGGCAGG
TGCCAGCACAGCGTCCTGGGTGACTTCAAGCCCTACAAGCCTGGCCAGGC
CTACGTTCCCTGCTACTTCATCCATGTGCTCAAGAAGACAGACTGAGTTT
CTCCGGCTCCCAGAAGCCCATGCTCAGGCAATGGCCCCTACCCTAAGACC
ATCCCCTAATGCAGATATTGCATTTGGGTGCAGATGTGGGGGTCGGGCAA
ACGGAGTAAACAATACAGTCTGCATTCTCCAAAAAAAAAAAAAAAAAAAA
AAAAAAAAAAAAAAAAAAAAAAAAAAAAAAAAAAAAAAA).

In some embodiments, a GNMT gene sequence is a nucleotide sequence that has at least 99%, 98%, 97%, 96%, 95%, 90%, 85%, 80%, 75%, 70%, 65%, 60%, 55%, or 50% homology with the nucleotide sequence shown in SEQ ID NO: 6. In some embodiments, a GNMT gene encodes a catalytically-active fragment of any of the GNMT polypeptides described above. In some embodiments, a GNMT gene comprises a portion of the nucleotide sequence shown in SEQ ID NO: 6 that encodes the polypeptide shown in SEQ ID NO: 5, or that encodes a polypeptide with at least 90% homology to the polypeptide shown in SEQ ID NO: 5.

### MTR Gene

Embodiments provided herein may involve a methionine synthase (also known as 5-methyltetrahydrofolate-homocysteine methyltransferase) (MTR) gene or polypeptide (e.g. in some embodiments, the expression or activity of MTR is inhibited). MTR catalyzes the regeneration of methionine from homocysteine. Exemplary MTR gene and polypeptide sequences are provided via GenBank Accession Nos. NP_001074597.1 (mouse) and NG_008959.1 (human).

### Nucleotide Sequence of Interest

Embodiments provided herein may include a nucleotide sequence of interest. As used herein, a “nucleotide sequence of interest” refers to any nucleotide sequence that a person may want to introduce into a host cell. Most commonly, a nucleotide sequence of interest is a DNA sequence that encodes a polypeptide of interest or that is a template for the generation of an RNA molecule of interest. However, a nucleotide sequence of interest may alternatively, for example, be a sequence which provides a regulatory or structural function (e.g. a promoter or enhancer sequence), or which serves a different purpose. A nucleotide sequence of interest may be of any nucleotide length. A nucleotide sequence of interest may be a DNA sequence or an RNA sequence. In some embodiments, a nucleotide sequence of interest is a sequence that is not endogenously present in the host cell. In some embodiments, a nucleotide sequence of interest is separately endogenously present in the host cell (i.e. the sequence is also present in the host cell separate from a recombinant nucleic acid construct containing the nucleotide sequence of interest introduced into the host cell). In such embodiments, the nucleotide sequence of interest may be introduced into a host cell, for example, if there is low expression of the corresponding endogenous nucleotide sequence, and it is desirable to have increased expression of the nucleotide sequence in the cell.

In some embodiments, a nucleotide sequence of interest encodes a polypeptide of interest (via transcription into mRNA and translation of the mRNA). Polypeptides of interest include, for example, an antibody, an enzyme, a peptide hormone, a fusion protein, or a detectable protein (e.g. a fluorescent protein such as a green fluorescent protein). In some embodiments, a polypeptide of interest may be a structurally or functionally defined part of a polypeptide, for instance, a fragment of an antibody, such as a heavy chain, light chain, or constant region of an antibody, or a catalytic domain of an enzyme. As understood by a person of skill in the art, a polypeptide may be of more than one of the types mentioned above (e.g. an enzyme may also be a detectable protein, etc.).

In some embodiments, a nucleotide sequence of interest is a DNA template for an RNA molecule of interest. RNA molecules of interest include, for example, CRISPR-cas9 system related RNA or RNAi (interfering RNA)-related molecules such as miRNA, siRNA, or shRNA. A “small interfering” or “short interfering RNA” or siRNA is a RNA duplex of nucleotides that is targeted to a gene interest or the one or more genes. An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length. A “short hairpin RNA,” or shRNA, is a polynucleotide construct that can be made to express an interfering RNA such as siRNA.

### Nucleic Acid Constructs

In some aspects, provided herein are nucleic acid constructs. A “nucleic acid construct” as provided herein is a type of polynucleotide or nucleic acid described above. A “nucleic acid construct” may have any of the characteristics of a polynucleotide or nucleic acid described above. Typically, a “nucleic acid construct” as provided herein contains two or more functional units within the chain of nucleotides that make up the polynucleotide. A functional unit in a nucleotide sequence may be any type of discrete nucleotide sequence having a particular function such as, for example, a nucleotide sequence of interest, a gene encoding a polypeptide, a regulatory sequence, a recombination sequence, or a template for an inhibitory RNA molecule. Thus, for example, some embodiments of a “nucleic acid construct” provided herein may contain one or more of the following:
i) a CBS, CTH, and/or GNMT gene;
ii) any number of nucleotide sequences of interest, such as 1, 2, 3, 4, 5, or more nucleotide sequences of interest;
iii) any number of recombination target sequences, such as 1, 2, 3, 4, 5, or more recombination target sequences;
iv) any number of expression control sequences, such as 1, 2, 3, 4, 5, or more expression control sequences. Optionally each nucleotide sequence of interest and CBS or CTH gene are operably linked to at least one expression control sequence.

A CBS gene, CTH gene, GNMT gene, nucleotide sequence of interest, or expression control sequence in a nucleic acid construct may have any of the respective properties described elsewhere herein. Also, as would be understood by a person of skill in the art, various features of a nucleic acid construct as listed above such as a CBS gene, CTH gene, recombination target site, or expression control sequence could also be considered as a “nucleotide sequence of interest”; however, these are separately noted at times herein in order to provide additional details about particular embodiments disclosed herein.

A “recombination target sequence” or a “recombination target site” is a stretch of nucleotides being necessary for and allowing, together with a recombinase, a targeted recombination and defining the location of such a recombination. As used herein, “recombination target sequence” is typically used to refer to a recombination sequence on an exogenous nucleic acid construct to be introduced into a host cell, and “recombination target site” is typically used to refer to a corresponding recombination sequence in a host cell chromosome. A recombination target site may be non-native to a host cell genome (e.g. it may be introduced into a host cell chromosome as part of a landing pad sequence).

In some embodiments, one or more recombination target sequences may be included in a nucleic acid construct provided herein, so that some or all of the nucleic acid construct may be integrated into a corresponding site at in a host cell chromosome.

Any suitable recombination target site/sequence and recombinase combination may be used with the compositions and methods provided herein, including both cysteine recombinase and serine recombinase-based systems. Recombinases (and their corresponding recombination target sequences) that may be used with nucleic acid constructs and host cells provided herein include, for example, Cre, Dre, Flp, KD, B2, B3, λ, HK022, HP1, γδ, ParA, Tn3, Gin, Bxb1, and R4. Site specific recombinases are described, for example, in Turan and Bode, The FASEB Journal, 25 (12): 4088-107 (2011); Nern et al, PNAS, 108 (34): 14198-203 (2011); and Xu et al, BMC Biotechnology, 13 (87) (2013).

In some embodiments, a recombination target sequence is a Flp recognition target (“FRT”) site (for use with a Flp recombinase). A FRT site may be a wild type FRT site (referred to sometimes as an “F site”) or a mutant FRT site, such as an “F5 site” as disclosed in Schlacke and Bode (1994) Biochemistry 33:12746-12752. In case the recombination target site is a FRT site, the host cells need the presence and expression of FLP (FLP recombinase) in order to achieve a cross-over or recombination event. The FRT site is a 34 base pair long nucleotide sequence which enables a site-directed recombination technology allowing the manipulation of an organism DNA under controlled conditions in vivo. The FRT is bound by the FLP recombinase which subsequently cleaves said sequence and allows the recombination of nucleotide sequences integrated between two FRT sites. For recombination mediated cassette exchange (“RMCE”), two cross-over events are required mediated by two flanking recombinase target sequences; one at the 5′ and one at the 3′ end of the cassette to be exchanged. A cross-over can occur between two identical FRT sites. The use of FRT sites also requires the expression and presence of the FLP recombinase. The whole system, herein also called “FRT/FLP”, is disclosed, for example, in Seibler and Bode, Biochemistry 36 (1997), pages 1740 to 1747, and Seibler et al., Biochemistry 37 (1998), pages 6229 to 6234.

In some embodiments, a recombination target sequence is a lox sequence (for use with the Cre recombinase). The lox site is 34 base pairs long, containing two 13 base pair palindromic sequences.

In some embodiments, a recombination target sequence is a sequence for use with a Bxb1 recombinase.

In order for a nucleic acid construct to be integrated into a host cell genome by a recombinase, the recombinase must be present in the host cell. The recombinase may be introduced into the host cell by any suitable method known in the art. For example, the recombinase may be encoded by a gene included on a nucleic acid construct provided herein, it may be encoded by a gene on a vector introduced into a host cell separate from a nucleic acid construct containing CBS and/or CTH genes, or it may be encoded by a gene stably integrated into the genome of the host cell (e.g. under the control of an inducible promoter). In some embodiments, a recombinase gene may be included in a recombinant nucleic acid construct containing one or more recombination target sites. In other embodiments, a recombinase gene may be introduced into a host cell in a nucleic acid separate from a recombinant nucleic acid construct containing one or more recombination target sites.

In some embodiments, a nucleic acid construct provided herein may contain 1, 2, 3, 4, 5, or more recombination target sequences. In some embodiments, a nucleic acid construct may contain a first recombination target sequence and a second recombination target sequence, wherein the first recombination target sequence and the second recombination target sequence flank (i.e. surround) the nucleotide sequences of interest and the CBS and/or CTH genes of the nucleic acid construct (if present). Put another way, in some embodiments, a nucleic acid construct may contain a first recombination target sequence and a second recombination target sequence and one or more of: i) one or more nucleotide sequences of interest; ii) a CBS gene, and iii) a CTH gene, and any of items i), ii), and iii), if present, are between the first recombination target sequence and the second recombination target sequence. Put yet another way, in some embodiments, a nucleic acid construct may contain a first recombination target sequence and a second recombination target sequence and one or more of: i) one or more nucleotide sequences of interest; ii) a CBS gene, and iii) a CTH gene, and the first recombination target sequence is 5′ to any of items i), ii), and iii), if present, and the second recombination target sequence is 3′ any of items i), ii), and iii), if present. In some embodiments, a nucleic acid construct may contain a first recombination target sequence and a second recombination target sequence and one or more of: i) one or more nucleotide sequences of interest; ii) a CBS gene, and iii) a CTH gene, and any of items i), ii), and iii), if present, are between the first recombination target sequence and the second recombination target sequence, such that items i), ii), and iii), if present, may be integrated into a targeted region of a host cell chromosome via recombination mediated cassette exchange (RMCE). In some embodiments, in a nucleic acid construct containing a first recombination target sequence and a second recombination target sequence, the first recombination target sequence is a wild-type FRT site and the second recombination target sequence is a mutant FRT site. A recombination target sequence in a nucleic acid construct may be located directly adjacent to or at a defined distance to a nucleotide sequence of interest, a CBS gene, or a CTH gene. In some embodiments, a recombination target sequence may be positioned in forward or reverse orientation. In a recombination nucleic acid containing a first recombination target sequence and a second recombination target sequence, in some embodiments, the first and second recombination target sequence are both in the forward or are both in the reverse orientation.

In some embodiments, a nucleotide sequence of interest (e.g. a gene encoding a polypeptide of interest) in a nucleic acid construct may be linked to one or more regulatory genetic control elements in the nucleic acid construct. In certain embodiments, a genetic control element directs constitutive expression of the nucleotide sequence of interest. In certain embodiments, a genetic control element that provides inducible expression of a nucleotide sequence of interest can be used. The use of an inducible genetic control element (e.g., an inducible promoter) allows for modulation of the production of, for example, a polypeptide encoded by a gene. Non-limiting examples of potentially useful inducible genetic control elements for use in eukaryotic cells include hormone-regulated elements (e.g., see Mader, S. and White, J. H., Proc. Natl. Acad. Sci. USA 90:5603-5607, 1993), synthetic ligand-regulated elements (see, e.g. Spencer, D. M. et al., Science 262:1019-1024, 1993) and ionizing radiation-regulated elements (e.g., see Manome, Y. et al., Biochemistry 32:10607-10613, 1993; Datta, R. et al., Proc. Natl. Acad. Sci. USA 89:10149-10153, 1992). Additional cell-specific or other regulatory systems known in the art may be used in accordance with the methods and compositions provided herein.

In some aspects, provided herein is a vector containing a nucleic acid construct. The nucleic acid construct may have any of the characteristics as described elsewhere herein.

In some embodiments a vector contains one or more of a promoter sequence, a directional cloning site, a non-directional cloning site, a restriction site, an epitope tag, a polyadenylation sequence, and antibiotic resistance gene. In some embodiments the promoter sequence is Human cytomegalovirus immediate early promoter, the directional cloning site is TOPO, the epitope tag is V5 for detection using anti-V5 antibodies, the polyadenylation sequence is from Herpes Simplex Virus thymidine kinase, and antibiotic resistance gene for is blasticidin, puromycin, or geneticin (G418).

In some embodiments provided herein, recombinant nucleic acid sequences such as promoter sequences, a directional cloning sites, sequences encoding epitope tags, polyadenylation sequences, antibiotic resistance genes, and protein coding genes may be part of both nucleic acid constructs and vectors.

In some embodiments, a vector provided herein is an expression vector. Expression vectors generally are replicable polynucleotide constructs that contain a recombinant nucleic acid construct according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

Polynucleotides provided herein may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials. Polynucleotides complementary to any nucleic acid construct or vector sequences provided herein are also encompassed by the present invention. It will be appreciated by those of ordinary skill in the art that, as a result of the degeneracy of the genetic code, there may be multiple nucleotide sequences that encode a polypeptide provided herein.

Homology analysis of polynucleotide or polypeptide sequences may be performed using methods known in the art (e.g. BLAST). Comparisons between two sequences are typically performed by comparing the sequences over a comparison window to identify and compare local regions of sequence similarity. Preferably, percent homology or sequence identity is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Polynucleotides provided herein can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g. without limitation, pUC18, pUC19, Bluescript (e.g., pBS SK+) and its derivatives, mp18, mp19, pBR322, pMB9, CoIE1, pCR1, RP4, phage DNAs, and shuttle vectors such as pSA3 and pAT28. These and many other cloning vectors are available from commercial vendors such as BioRad, Strategene, and Invitrogen.

### Host Cells

As used herein, the term “host cell”, refers to a cell or cell culture harboring a recombinant nucleic acid construct provided herein, or that can be a recipient for such nucleic acid constructs. Host cells include progeny of a single host cell, and the progeny may not necessarily be completely identical (in morphology or in genomic DNA complement) to the original parent cell due to natural, accidental, or deliberate mutation.

In some embodiments, a host cell may harbor the recombinant nucleic acid construct stably integrated at a location in its genome (e.g. in a chromosome). In some embodiments, a recombinant nucleic acid construct in a host cell is not stably integrated into the host cell's genome—e.g. the recombinant nucleic acid construct may be in the host cell in a plasmid.

In the context of the present disclosure, a “cell” is preferably a mammalian cell. A mammalian cell may be, for example, a canine cell (e.g. Madin-Darby canine kidney epithelial (MDCK) cell), a primate cell, a human cell (e.g. human embryonic kidney (HEK) cell), a mouse cell or a hamster cell. In some embodiments, a hamster cell is a Chinese hamster ovary (CHO) cell. Optionally, a CHO cell may be a CHOK1 or a CHOK1 SV cell (Porter, A J et al. Biotechnol Prog. 26 (2010), 1455-1464). In some embodiments, a mammalian cell is a BALB/c mouse myeloma cell, a human retinoblast cell (PER.C6), a monkey kidney cell, a human embryonic kidney cell (293), a baby hamster kidney cell (BHK), a mouse sertoli cell, an African green monkey kidney cell (CERO-76), a HeLa cell, a buffalo rat liver cell, a human lung cell, a human liver cell, a mouse mammary tumor cell, a TRI cell, a MRC 5 cell, a FS4 cell, or a human hepatoma cell (e.g. Hep G2). In some embodiments, a cell is a non-mammalian cell (e.g. an insect cell or a yeast cell).

Embodiments of the present disclosure are particularly suited for use with mammalian cells that are cysteine auxotrophs (i.e. which are cysteine auxotrophs, absent the introduction into the cell of one or more recombinant nucleic acid constructs provided herein). As used herein, in the context of a particular nutrient, an “auxotroph” refers a cell that requires that nutrient from outside the cell for normal growth/survival (i.e. the cell cannot synthesize sufficient amounts of that nutrient for normal functioning). In contrast, as used herein, in the context of a particular nutrient, a “prototroph” refers to a cell that can synthesize sufficient quantities of that nutrient for normal growth/survival (i.e. the cell can synthesize sufficient amounts of that nutrient for normal functioning). Thus, for example, a “cysteine auxotroph” refers to a cell that cannot synthesize sufficient quantities of cysteine for normal functioning. Accordingly, a cell which is a “cysteine auxotroph” must receive cysteine from a source outside the cell for proper growth; typically, this is achieved by culturing a cell which is a cysteine auxotroph in a cysteine-containing cell culture medium. In contrast, a cell which is a “cysteine prototroph” does not need to receive cysteine from a source outside of the cell, and thus, a cysteine prototroph cell may, for example, be cultured in cell culture medium that does not contain cysteine (or which only contains very low concentrations of cysteine). As would be understood by a person of skill in the art, in some embodiments, a “cysteine auxotroph” may still have some growth or survival in a cysteine-deficient medium, but that growth or survival is significantly less than would occur in a cell culture medium containing a sufficient quantity of cysteine (i.e. the cells are distressed).

As an example, CHO cells are cysteine auxotrophs. In some embodiments, methods and compositions provided herein may be used with any cell line which is a cysteine auxotroph. In some embodiments, cell lines that are cysteine auxotrophs may be identified by assaying the cell line for growth in a cysteine-deficient medium. [Optionally, growth of a cell line in cysteine-deficient media may be assayed preparing two versions of an appropriate medium for the cell line, in which the two versions are identical except for the first version of the medium contains a standard amount of cysteine (e.g. optionally around, for example, 2-5 mM cysteine) for the medium, and the second version of the medium contains little or no cysteine (and optionally, at least 50%, 60%, 70%, 80%, 90%, or 95% less cysteine than the first version of the medium) (e.g. less than about 2 mM, less than about 1.8 mM, less than about 1.6 mM, less than about 1.5 mM, less than about 1.6 mM, less than about 1.4 mM, less than about 1.2 mM, less than about 1 mM, less than about 0.8 mM, less than about 0.6 mM, less than about 0.5 mM, less than about 0.2 mM, less than about 0.1 mM, less than about 50 μM, less than about 20 μM, less than about 10 μM, less than about 5 μM, less than about 2 μM, less than about 1 μM, or 0 M cysteine). The cell line to be tested can then be cultured in the different versions media under otherwise identical conditions; a cell line which has significantly impaired growth in the cysteine-deficient medium as compared to the standard cysteine-containing medium is a cysteine auxotroph.] In some embodiments, cell lines that are cysteine auxotrophs may be identified by assaying the expression levels of the CBS and CTH genes in the cell line; cells with low expression levels of CBS and CTH genes are generally cysteine auxotrophs (this can be confirmed by testing the growth of the respective cell line in a cysteine-deficient medium as described above).

In some embodiments, a cell or cell culture that has “significantly impaired growth” (or the like) in a second cell culture medium as compared to in a first cell culture medium will have a doubling time in the second cell culture medium which is at least about 10%, 20%, 30%, 40%, 50%, 60%, 70%, 80%, 90%, or 100% greater than in the first cell culture medium (i.e. it takes a more time to double in the second cell culture medium), when the cells are otherwise cultured under the same conditions. In some embodiments, a cell or cell culture that has “significantly impaired growth” in a second cell culture medium as compared to in a first cell culture medium will have a cell count in the second cell culture medium which is at least about 10%, 20%, 30%, 40%, 50%, 60%, 70%, 80%, 90%, or 100% less than in the first cell culture medium, when the cells are otherwise cultured under the same conditions for the same period of time. In some embodiments, a cell or cell culture that has “significantly impaired growth” in a second cell culture medium as compared to in a first cell culture medium will have a cell density in the second cell culture medium which is at least about 10%, 20%, 30%, 40%, 50%, 60%, 70%, 80%, 90%, or 100% less than in the first cell culture medium, when the cells are otherwise cultured under the same conditions for the same period of time. Optionally, the descriptions provided above for comparing cell or cell culture growth in a first and second cell culture medium, may similarly apply for comparing cell or cell culture growth under different culture conditions (e.g. different temperatures, etc.).

In some embodiments, methods using the cysteine selection marker system provided herein may be used with host cells that are derived from a parental cell line that originally was a cysteine prototroph, but which was genetically modified to be converted to a cysteine auxotroph. For instance, in some embodiments, a cell that is a cysteine prototroph may be converted to a cysteine auxotroph by deleting or mutating one or more genes in the cysteine metabolism pathway in the cell (e.g. the CBS and CTH genes).

Genes in a cell may be deleted or mutated by methods known in the art, such as by CRISPR, TALEN, or zinc-finger related processes.

In some other embodiments, a cell that is a cysteine prototroph may be converted to a cysteine auxotroph by deleting or mutating one or more genes in the cell in the cysteine metabolism pathway selected from, for example, BHMT (betaine-homocysteine methyltransferase); BHMT2 (betaine-homocysteine methyltransferase 2); MTR (5-methyltetrahydrofolate-homocysteine methyltransferase); AHCY (S-adenosylhomocysteine hydrolase); MAT1A (methionine adenosyltransferase I, alpha); MAT2A (methionine adenosyltransferase II, alpha); MAT2B (methionine adenosyltransferase II, beta); DNMT1 (DNA methyltransferase (cytosine-5) 1); DNMT3A (DNA methyltransferase 3A); DNMT3B (DNA methyltransferase 3B), KYAT1, KYAT3, AHCYL1, and AHCYL2. In some embodiments, methods and compositions as provided herein for use with the CBS and CTH genes may be used with one or more of the genes provided above and a corresponding host cell that has had the respective gene(s) deleted or mutated in the host cell. For example, in some embodiments, provided herein is a nucleic acid construct comprising a nucleotide sequence of interest and an AHCY gene; also provided herein is a host cell comprising the nucleic acid construct, wherein the host cell has had the endogenous AHCY gene mutated or deleted.

In some embodiments, a host cell that has received recombinant CBS and CTH genes according to methods and compositions provided herein may have a greater ability to proliferate in a cysteine-deficient cell culture medium than a corresponding cell that does not contain the recombinant CBS and CTH genes. Cell proliferation may be measured, for example, by measuring DNA synthesis in the cells (e.g. by assaying for labeled DNA), by assaying cellular metabolism, by assaying proliferation markers (e.g. for Ki-67), by measuring cell growth rates (e.g. doubling time), or by measuring cell density or numbers. A cell or cell culture that has a “greater ability to proliferate” (or the like) than a corresponding cell/cell culture will have greater values (or, where appropriate, a smaller value, where the smaller value indicates faster growth) for at least one, two, or three of the above characteristics than the corresponding cell/culture over which it has a “greater ability to proliferate”. In some embodiments, a cell or cell culture that has a “greater ability to proliferate” than a corresponding cell/cell culture will have a doubling time which is at least about 10%, 20%, 30%, 40%, 50%, 60%, 70%, 80%, 90%, or 100% less than the corresponding cell/cell culture (i.e. the cell or cell culture with a greater ability to proliferate doubles in less time than the corresponding cell or cell culture), when the cells are cultured under the same conditions for the same period of time. In some embodiments, a cell or cell culture that has a “greater ability to proliferate” than a corresponding cell/cell culture will have a cell count which is at least about 10%, 20%, 30%, 40%, 50%, 60%, 70%, 80%, 90%, or 100% greater than the corresponding cell/cell culture, when the cells are cultured under the same conditions for the same period of time. In some embodiments, a cell or cell culture that has a “greater ability to proliferate” than a corresponding cell/cell culture will have a cell density which is at least about 10%, 20%, 30%, 40%, 50%, 60%, 70%, 80%, 90%, or 100% greater than the corresponding cell/cell culture, when the cells are cultured under the same conditions for the same period of time. As used herein, a cell that has a “greater ability to proliferate” may also be described as having “improved growth characteristics”, and the like.

In some aspects, also provided herein are host cells that have received one or more nucleic acid constructs that contain the CBS and CTH genes but which do not contain a nucleotide sequence of interest encoding, for example, a polypeptide of interest or RNA molecule of interest. Also are provided herein are related compositions and methods of making the cells. In some embodiments, such host cells (e.g. which contain an exogenously-introduced CBS and CTH gene and as a result have higher CBS and CTH expression than a corresponding host cell that has not received CBS and CTH-containing constructs) may be of interest, for example, for their ability to grow in cysteine-deficient media. Use of cysteine-deficient media may simply media preparation and/or lower media cost.

In some additional aspects, also provided herein are host cells that have not received exogenous CBS and/or CTH genes, but which have been genetically modified such that their endogenous CBS and/or CTH genes have higher expression than in corresponding non-modified cells. For example, in some embodiments, a recombinant promoter sequence may be introduced into a host cell genome such that, once it is introduced, it is operably linked to the endogenous CBS or CTH gene, and causes increased expression of the respective endogenous CBS or CTH gene. Host cells which are modified to have increased expression of their endogenous CBS and CTH genes may be useful, for example, for their ability to proliferate in cysteine-deficient media (e.g. for the reasons described above).

Thus, in some embodiments, the methods and compositions described above (in which the expression of the CBS and CTH gene is increased in a host cell without necessarily also introducing into the host cell an exogenous nucleotide sequence of interest encoding a polypeptide of interest or RNA sequence of interest) may be used with any host cell which in its unmodified form has a low expression level of the CBS and CTH genes. These methods and compositions may be used to modify such host cells to, for example, reduce or eliminate the need for such cells to have cysteine in media for the cells.

Introduction of Polynucleotides into Cells

Polynucleotides provided herein (e.g. nucleic acid constructs, vectors, etc.) can be introduced into a host cell by any of a number of appropriate means, including, for example, electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of method for introduction of a polynucleotide into a host cell will often depend on features of the host cell.

Methods suitable for introducing nucleic acids sufficient to achieve expression of a protein of interest into mammalian host cells are known in the art. See, for example, Gething et al., Nature, 293:620-625, 1981; Mantei et al., Nature, 281:40-46, 1979; Levinson et al. EP 117,060; and EP 117,058, each of which is incorporated herein by reference. For mammalian cells, common methods of introducing genetic material into mammalian cells include the calcium phosphate precipitation method of Graham and van der Erb (Virology, 52:456-457, 1978) or the Lipofectamine™ (Gibco BRL) Method of Hawley-Nelson (Focus 15:73, 1993). General aspects of mammalian cell host system transformations have been described by Axel in U.S. Pat. No. 4,399,216 issued Aug. 16, 1983. For various techniques for introducing genetic material into mammalian cells, see Keown et al., Methods in Enzymology, 1989, Keown et al., Methods in Enzymology, 185:527-537, 1990, and Mansour et al., Nature, 336:348-352, 1988. Additional methods suitable for introducing nucleic acids include electroporation, for example as employed using the GenePulser XCeII™ electroporator by BioRad™—Non-limiting representative examples of suitable vectors for expression of proteins in mammalian cells include pCDNA1; pCD, see Okayama, et al. Mol. Cell Biol. 5:1136-1142, 1985; pMClneo Poly-A, see Thomas, et al. Cell 51:503-512, 1987; a baculovirus vector such as pAC 373 or pAC 610; CDM8, see Seed, B. Nature 329:840, 1987; and pMT2PC, see Kaufman, et aL. EMBO J. 6:187-195, 1987, each of which is incorporated herein by reference in its entirety.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther., 1992, 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther., 1992, 3:147); ligand-linked DNA (see, e.g., Wu, J. Biol. Chem., 1989, 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes.

Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol., 1994, 14:2411, and in Woffendin, Proc. Natl. Acad. Sci., 1994, 91:1581. Naked DNA can be introduced into cells by forming a precipitate containing the DNA and calcium phosphate.

Alternatively, naked DNA can also be introduced into cells by forming a mixture of the DNA and DEAE-dextran and incubating the mixture with the cells or by incubating the cells and the DNA together in an appropriate buffer and subjecting the cells to a high-voltage electric pulse (e.g., by electroporation). Naked DNA can also be directly injected into cells by, for example, microinjection. Alternatively, naked DNA can also be introduced into cells by complexing the DNA to a cation, such as polylysine, which is coupled to a ligand for a cell-surface receptor (see for example Wu, G. and Wu, C. H. J. Biol. Chem. 263:14621, 1988; Wilson et al. J. Biol. Chem. 267:963-967, 1992; and U.S. Pat. No. 5,166,320, each of which is hereby incorporated by reference in its entirety). Binding of the DNA-ligand complex to the receptor facilitates uptake of the DNA by receptor-mediated endocytosis.

In certain embodiments, a polynucleotide provided herein is stably introduced into a host cell. In certain embodiments, a polynucleotide provided herein is transiently introduced into the host cell.

Integration of Nucleic Acids into Host Cell Genomes

In embodiments provided herein in which a polynucleotide is stably introduced into a host cell (for example, in situations where the polynucleotide is integrated into a host cell chromosome), the polynucleotide may be randomly integrated into a chromosome in the host cell, or the polynucleotide may be integrated at a specific location in a chromosome in the host cell. These approaches may be referred herein to as a “random integration” or “site-specific integration (“SSI”)”, respectively.

For random integration, typically, one or more recombinant nucleic acid constructs are prepared in which the recombinant nucleic acid construct(s) each contain at least one nucleotide sequence of interest and at least one gene that is all or part of a selectable marker system. For example, the cysteine selectable marker system provided herein comprises the CBS gene and CTH gene. For a cysteine auxotroph cell to be converted to a cysteine prototroph, the cysteine auxotroph cell receives exogenous copies of both the CBS gene and CTH gene. The CBS gene and the CTH gene may be introduced into a host cell on separate exogenous nucleic acid constructs, or together on the same exogenous nucleic acid construct. Accordingly, in some embodiments provided herein, a recombinant nucleic acid construct containing a first nucleotide sequence of interest and at least one of the CBS gene and CTH gene is provided. In some embodiments, a recombinant nucleic acid construct containing the nucleotide sequence of interest and both the CBS gene and the CTH gene is provided. In some embodiments, a first recombinant nucleic acid construct containing a first nucleotide sequence of interest and the CBS gene, and a second recombinant nucleic acid construct containing a second nucleotide sequence interest and the CTH gene are provided.

After preparation of the polynucleotide(s) containing the genes of the cysteine selectable marker system, the polynucleotides are introduced into a population of cysteine auxotroph cells, and cells in which the polynucleotide(s) have integrated are selected for by growth of the cells in cysteine-deficient media. Generally, after polynucleotide(s) containing the genes of the cysteine selectable marker system are introduced into a population of cysteine auxotroph cells, and cells are selected for by growth in a cysteine-deficient medium, there may be a heterogeneous population of cells (also referred to herein as a “pool” of cells) containing different numbers of copies of the polynucleotide(s) containing the CBS and CTH genes in the cell, as well as different locations of integration of the polynucleotide(s) in chromosomes in the cell. Optionally, individual cells from this pool of generated cysteine prototrophs may be sorted and isolated, and individual homogenous cell line populations of different cysteine prototrophs may be established (also referred to herein as cell line “clones”). Different clones of cysteine prototrophs may exhibit, for example, different levels of protein production of a gene encoding a polypeptide of interest (if present) on a nucleic acid construct containing the CBS and/or CTH gene, or different cell growth rates. Alternatively, in some embodiments, a heterogeneous pool of exogenous CBS and CTH gene-containing cells may be maintained, and used for various methods (e.g. protein production) as described herein.

In some embodiments, nucleic acid constructs for random integration may be linear polynucleotides. In some embodiments, the linear structure may be generated by synthesis of a linear molecule (e.g. by PCR or chemical polynucleotide synthesis). In some embodiments, the linear structure may be generated by cleavage of a circular vector (e.g. by a restriction enzyme) to generate a linear nucleic acid molecule.

In some embodiments, provided herein is a host cell comprising one or more nucleic acid constructs provided herein integrated into a chromosome of the cell. For example, in some embodiments, provided herein is a host cell comprising a recombinant nucleic acid construct comprising a nucleotide sequence of interest, a CBS gene, and a CTH gene integrated into a chromosome of the cell. In another example, in some embodiments, provided herein is a host cell comprising a first recombinant nucleic acid construct comprising a first nucleotide sequence of interest and a CBS gene integrated into a chromosome of the cell, and a second recombinant nucleic acid construct comprising a second nucleotide sequence of interest and a CTH gene integrated into a chromosome of the cell, wherein the chromosome containing the first recombinant nucleic acid construct and the chromosome containing the second recombinant nucleic acid construct may be the same or different chromosomes.

For site-specific integration, in some embodiments, a host cell that contains a “landing pad” at a defined chromosomal locus is used. The landing pad contains an exogenous nucleotide sequence that contains one or more recombination target sites, which is stably integrated into a chromosome. When an exogenous nucleic acid construct that contains one or more recombination target sequences that correspond to the recombination target site in the landing pad is introduced into the host cell, an expression cassette in the exogenous nucleic acid construct may be integrated into or replace the landing pad sequence (for example, via recombinase mediated cassette exchange (RMCE)). In embodiments, the cysteine selectable marker system as provided herein may be used with an SSI system as described, for example, in Zhang L, et. al (Biotechnol Prog. 2015; 31: 1645-1656) or International Publication WO 2013/190032, which are hereby incorporated by reference for all purposes.

In some embodiments, a landing pad in a host cell line may be located at a “hot-spot” in the host cell's genome. As used herein, the term “hot-spot” means a site, in the genome of a host cell which provides for a stable and high expression of a gene or genes integrated at the site.

A cell that contains a landing pad for SSI may also be referred to herein as a “SSI host cell”. As used herein, “SSI host cell” refers to a host cell that contains an exogenous nucleotide sequence that includes at least one recombination target site (e.g. a landing pad). The recombination target site in the host cell permits site specific integration of exogenous nucleotide sequences into the genome of the host cell, thus enabling a predetermined localized and directed integration of desired nucleotide sequences at a desired place in a host cell's genome. Thus, in some embodiments, a site-specific integration host cell is capable of targeted integration of a recombinant nucleic acid construct (or an expression cassette therein) described herein into a chromosome of the host cell. In some embodiments, a site-specific integration host cell is capable of targeted integration of an expression cassette by recombination mediated cassette exchange (RMCE).

For compositions and methods provided herein involving recombination of an exogenous nucleic acid construct into a host cell genome, as described above, a recombinase is also present or introduced into the host cell. Methods provided herein involving introducing an exogenous nucleic acid construct may include introducing a gene encoding a recombinase into the host cell.

In some embodiments, a cysteine selectable marker system as used herein may be used to select for cells that have received at a specific chromosomal location one or more polynucleotide cassettes, each cassette containing a polynucleotide sequence of interest and one or both of the CBS and CTH genes. For example in some embodiments provided herein for SSI, a recombinant nucleic acid construct containing an expression cassette containing a first nucleotide sequence of interest and at least one of the CBS gene and CTH gene is provided. In some embodiments provided herein for SSI, a recombinant nucleic acid construct containing an expression cassette containing the nucleotide sequence of interest and both the CBS gene and the CTH gene is provided. In some embodiments provided herein for SSI, a first recombinant nucleic acid construct containing a first expression cassette contains a first nucleotide sequence of interest and the CBS gene, and a second recombinant nucleic acid construct containing a second expression cassette containing a second nucleotide sequence interest and the CTH gene are provided. Optionally, the first expression cassette and the second expression cassette may be flanked by recombination target sites for a first SSI location and a second SSI location, respectively, such that the first expression cassette and second expression cassette are targeted for integration into different chromosomal locations in the host cell (e.g. a first chromosomal locus and a second chromosomal locus).

In some embodiments, provided herein is a host cell comprising an exogenous recombinant nucleic acid construct integrated into a specific location in a chromosome in the cell. The nucleic acid construct may have any of the properties of a nucleic acid construct provided herein, and may contain, for example a nucleotide sequence of interest and a CBS gene and a CTH gene. In some embodiments, provided herein is a host cell comprising one or more nucleic acid constructs provided herein integrated into a specific location/landing pad in a chromosome of the cell. For example, in some embodiments, provided herein is a host cell comprising a recombinant nucleic acid construct comprising a nucleotide sequence of interest, a CBS gene, and a CTH gene integrated into specific location in a chromosome in the cell. In another example, in some embodiments, provided herein is a host cell comprising a first recombinant nucleic acid construct comprising a first nucleotide sequence of interest and a CBS gene integrated into a first locus in a chromosome of the cell, and a second recombinant nucleic acid construct comprising a second nucleotide sequence of interest and a CTH gene integrated into a second locus in a chromosome of the cell, wherein the chromosome containing the first recombinant nucleic acid construct and the chromosome containing the second recombinant nucleic acid construct may be the same or different chromosomes.

### Recombinant Polypeptides

In another aspect, provided herein are recombinant polypeptides that are produced via the compositions and methods provided herein. For example, provided herein is a recombinant polypeptide that is encoded by a nucleotide sequence of interest that is a component of a recombinant nucleic acid construct provided herein.

Any polypeptide that is expressible in a host cell may be produced in accordance with the present teachings and may be produced according to the methods of the invention or by the cells of the invention. The polypeptide may have an amino acid sequence that occurs in nature, or may alternatively have a sequence that was engineered or selected by humans.

Polypeptides that may desirably be expressed in accordance with the present invention will often be selected on the basis of an interesting or useful biological or chemical activity. For example, the present invention may be employed to express any pharmaceutically or commercially relevant enzyme, receptor, antibody, hormone, regulatory factor, antigen, binding agent, etc. In some embodiments, the protein expressed by cells in culture are selected from antibodies, or fragments thereof, nanobodies, single domain antibodies, glycoproteins, therapeutic proteins, growth factors, clotting factors, cytokines, fusion proteins, pharmaceutical drug substances, vaccines, enzymes, or Small Modular ImmunoPharmaceuticals™ (SMIPs). One of ordinary skill in the art will understand that any protein may be expressed in accordance with the present invention and will be able to select the particular protein to be produced based on his or her particular needs.

### Antibodies

Given the large number of antibodies currently in use or under investigation as pharmaceutical or other commercial agents, production of antibodies is of particular interest in accordance with the present invention. Antibodies are proteins that have the ability to specifically bind a particular antigen. Any antibody that can be expressed in a host cell may be produced in accordance with the present invention and may be produced according to the methods of the invention or by the cells of the invention.

In embodiments provided herein involving a first nucleotide sequence of interest and a second nucleotide sequence of interest, optionally, the first nucleotide sequence of interest may encode a first polypeptide comprising an antibody variable heavy (VH) region, and the second nucleotide sequence interest may encode a second polypeptide comprising an antibody variable light (VL) region. Optionally, the first nucleotide sequence of interest may encode a polypeptide comprising an antibody heavy chain and the second nucleotide sequence of interest may encode a polypeptide comprising an antibody light chain. Optionally, the first nucleotide sequence of interest may encode a polypeptide comprising 3 CDRs of an antibody heavy chain and the second nucleotide sequence of interest may encode a polypeptide comprising 3 CDRs of an antibody light chain.

In some embodiments, an antibody produced according to the disclosure herein is a monoclonal antibody. In some embodiments, the monoclonal antibody is a chimeric antibody. A chimeric antibody contains amino acid fragments that are derived from more than one organism. Chimeric antibody molecules can include, for example, an antigen binding domain from an antibody of a mouse, rat, or other species, with human constant regions. A variety of approaches for making chimeric antibodies have been described. See e.g., Morrison et al., Proc. Natl. Acad. Sci. U.S.A. 81, 6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B.

In some embodiments, the monoclonal antibody is a human antibody derived, e.g., through the use of ribosome-display or phage-display libraries (see, e.g., Winter et al., U.S. Pat. No. 6,291,159 and Kawasaki, U.S. Pat. No. 5,658,754) or the use of xenographic species in which the native antibody genes are inactivated and functionally replaced with human antibody genes, while leaving intact the other components of the native immune system (see, e.g., Kucherlapati et al., U.S. Pat. No. 6,657,103).

In some embodiments, the monoclonal antibody is a humanized antibody. A humanized antibody is a chimeric antibody wherein the large majority of the amino acid residues are derived from human antibodies, thus minimizing any potential immune reaction when delivered to a human subject. In humanized antibodies, amino acid residues in the complementarity determining regions are replaced, at least in part, with residues from a non-human species that confer a desired antigen specificity or affinity. Such altered immunoglobulin molecules can be made by any of several techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and are preferably made according to the teachings of PCT Publication WO92/06193 or EP 0239400, all of which are incorporated herein by reference). Humanized antibodies can be commercially produced by, for example, Scotgen Limited, 2 Holly Road, Twickenham, Middlesex, Great Britain. For further reference, see Jones et al., Nature 321:522-525 (1986); Riechmann et al., Nature 332:323-329 (1988); and Presta, Curr. Op. Struct. Biol. 2:593-596 (1992), all of which are incorporated herein by reference.

In some embodiments, the monoclonal, chimeric, or humanized antibodies described above may contain amino acid residues that do not naturally occur in any antibody in any species in nature. These foreign residues can be utilized, for example, to confer novel or modified specificity, affinity or effector function on the monoclonal, chimeric or humanized antibody. In some embodiments, the antibodies described above may be conjugated to drugs for systemic pharmacotherapy, such as toxins, low-molecular-weight cytotoxic drugs, biological response modifiers, and radionuclides (see e.g., US20040082764 A1).

### Isolation of the Expressed Protein

In general, it will typically be desirable to isolate and/or purify proteins expressed according to the present invention. In certain embodiments, the expressed protein is secreted into the medium and thus cells and other solids may be removed, as by centrifugation or filtering for example, as a first step in the purification process. Alternatively, the expressed protein may remain in the cell or may be bound to the surface of the host cell. In such circumstances, the media may be removed and the host cells expressing the protein are lysed as a first step in the purification process. Lysis of mammalian host cells can be achieved by any number of means well known to those of ordinary skill in the art, including physical disruption by glass beads and exposure to high pH conditions.

The expressed protein may be isolated and purified by standard methods including, but not limited to, chromatography (e.g., ion exchange, affinity, size exclusion, and hydroxyapatite chromatography), gel filtration, centrifugation, or differential solubility, ethanol precipitation and/or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol. 182), Academic Press, 1997, each of which is incorporated herein by reference). For immunoaffinity chromatography in particular, the protein may be isolated by binding it to an affinity column comprising antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin may be added at any or all stages in order to reduce or eliminate degradation of the protein during the purification process. Protease inhibitors are particularly advantageous when cells must be lysed in order to isolate and purify the expressed protein.

One of ordinary skill in the art will appreciate that the exact purification technique will vary depending on the character of the protein to be purified, the character of the cells from which the protein is expressed, and/or the composition of the medium in which the cells were grown.

### Cell Cultures and Cell Culture Media

The terms “medium”, “media”, and the like as used herein refer to a solution containing components or nutrients which nourish growing mammalian cells. Typically, the nutrients include essential and non-essential amino acids, vitamins, energy sources, lipids, and trace elements required by the cell for minimal growth and/or survival. Such a solution may also contain further nutrients or supplementary components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), inorganic compounds present at high final concentrations (e.g., iron), amino acids, lipids, and/or glucose or other energy source. In some embodiments, a medium is advantageously formulated to a pH and salt concentration optimal for cell survival and proliferation. In some embodiments, a medium is a feed medium that is added after the beginning of the cell culture.

A wide variety of mammalian growth media may be used in accordance with the present invention. In some embodiments, cells may be grown in one of a variety of chemically defined media, wherein the components of the media are both known and controlled. In some embodiments, cells may be grown in a complex medium, in which not all components of the medium are known and/or controlled.

Chemically defined growth media for mammalian cell culture have been extensively developed and published over the last several decades. All components of defined media are well characterized, and so defined media do not contain complex additives such as serum or hydrolysates. Early media formulations were developed to permit cell growth and maintenance of viability with little or no concern for protein production. More recently, media formulations have been developed with the express purpose of supporting highly productive recombinant protein producing cell cultures. Such media are preferred for use in the method of the invention. Such media generally comprises high amounts of nutrients and in particular of amino acids to support the growth and/or the maintenance of cells at high density. If necessary, these media can be modified by the skilled person for use in the method of the invention. For example, the skilled person may decrease the amount of phenylalanine, tyrosine, cysteine, tryptophan and/or methionine in these media for their use as base media or feed media in a method as disclosed herein.

In some embodiments, provided herein are cysteine-deficient media. As used herein, “cysteine-deficient medium” and the like refers to a medium that does not contain enough cysteine to the support the normal growth and maintenance of cysteine auxotrophs (e.g. it does not support the growth and maintenance of cysteine auxotrophs at high density.) Cysteine auxotrophs have limited or no growth in cysteine-deficient media; accordingly, a cysteine-deficient media acts as a selective pressure for cysteine prototrophs.

As used herein, the reference to cysteine in the phrase “cysteine-deficient medium” (and the like) refers to the effective cysteine concentration in the medium. As used herein “effective cysteine concentration” of a solution (e.g. a cell culture medium) refers to the cysteine concentration of the solution, as calculated by taking into account the concentrations of both A) cysteine and B) cystine in the solution. Cystine is included in the “effective cysteine concentration” because it is an oxidized dimer of cysteine. Thus, the effective cysteine concentration of a solution is calculated as follows: effective cysteine concentration of a solution=[molar concentration cysteine in the solution+(2× molar concentration cystine in the solution)]. Thus, for example, a solution that contains 0.5 mM cysteine and 0.3 mM cystine has an effective cysteine concentration of 1.1 mM cysteine [0.5 mM (cysteine)+0.6 mM (cystine; 2×0.3 mM). Also, references herein to the “cysteine concentration” of a medium refer to the effective cysteine concentration of the medium, unless the context clearly dictates otherwise. Similarly, references herein to particular concentrations of cysteine in a medium (e.g. a medium that comprises “less than about 0.1 mM cysteine”) refer to the effective cysteine concentration in the solution (i.e. cysteine+cystine), unless the context clearly dictates otherwise.

In some embodiments, a cysteine-deficient medium provided herein contains less than about 2 mM, less than about 1.8 mM, less than about 1.6 mM, less than about 1.5 mM, less than about 1.6 mM, less than about 1.4 mM, less than about 1.2 mM, less than about 1 mM, less than about 0.8 mM, less than about 0.6 mM, less than about 0.5 mM, less than about 0.2 mM, less than about 0.1 mM, less than about 50 μM, less than about 20 μM, less than about 10 μM, less than about 5 μM, less than about 2 μM, less than about 1 μM, or 0 μM cysteine. In some embodiments, a cysteine-deficient medium contains about 1 mM cysteine or less, about 0.5 mM cysteine or less, about 0.2 mM cysteine or less, about 0.1 mM cysteine or less, about 50 μM cysteine or less, about 20 μM cysteine or less, about 10 μM cysteine or less, about 5 μM cysteine or less, about 2 μM cysteine or less, about 1 μM cysteine or less, or 0 M cysteine. Optionally, a cysteine-deficient medium is cysteine-free.

In an example, CHO cells are commonly cultured in a medium that contains at least about 2 mM cysteine; thus, in some embodiments, a CHO cell cultured in a cysteine-deficient medium is cultured in a medium containing less than about 2 mM, less than about 1.8 mM, less than about 1.6 mM, less than about 1.5 mM, less than about 1.6 mM, less than about 1.4 mM, less than about 1.2 mM, less than about 1 mM, less than about 0.8 mM, less than about 0.6 mM, less than about 0.5 mM, less than about 0.2 mM, less than about 0.1 mM, less than about 50 μM, less than about 20 μM, less than about 10 μM, less than about 5 μM, less than about 2 μM, less than about 1 μM, or 0 μM cysteine.

In some embodiments, a cysteine-deficient medium provided herein may further comprise low concentrations of one or more molecules which can be converted to cysteine (i.e. low concentrations of cysteine-related molecules other than cysteine and cystine). For example, in some embodiments, a cysteine-deficient medium provided herein further comprises a low concentration of glutathione (e.g. less than about 1 mM, less than about 0.8 mM, less than about 0.6 mM, less than about 0.5 mM, less than about 0.2 mM, less than about 0.1 mM, less than about 50 μM, less than about 20 μM, less than about 10 μM, less than about 5 μM, less than about 2 μM, less than about 1 μM or 0 glutathione). Each glutathione molecule contains a cysteine molecule (linked to a glutatamate and a glycine), and thus, in some situations, glutathione molecules may be converted to cysteine and affect the cysteine concentration of a solution. In some embodiments, a cysteine-deficient medium may further comprise a low concentration of reduced glutathione (GSH) or oxidized glutathione (GSSG).

Compositions and methods provided herein may be used with cysteine-deficient media, for example, to select for cells that contain nucleic acid constructs provided herein, wherein the nucleic acid constructs contain the genes of the cysteine selection marker system provided herein (i.e. CBS and CTH). Various media as described herein may be prepared in a cysteine-deficient format (i.e. in which the media has the various characteristics described herein, but with no cysteine or a low level of cysteine).

In some embodiments, host cells as provided herein which contain an increased expression or copy number of CBS and CTH genes may be used for their ability to efficiently grow in cysteine-deficient media. In some embodiments, it may be desirable to culture cells in cysteine-deficient in media, for example, in order to reduce the cost of the media, to simplify the preparation of the media, or to reduce any negative effects caused by the presence of cysteine in the media.

Not all components of complex media are well characterized, and so complex media may contain additives such as simple and/or complex carbon sources, simple and/or complex nitrogen sources, and serum, among other things. In some embodiments, complex media suitable for the present invention contains additives such as hydrolysates in addition to other components of defined medium as described herein.

In some embodiments, defined media typically includes roughly fifty chemical entities or components at known concentrations in water. Most of them also contain one or more well-characterized proteins such as insulin, IGF-1, transferrin or BSA, but others require no protein components and so are referred to as protein-free defined media. Typical chemical components of the media fall into five broad categories: amino acids, vitamins, inorganic salts, trace elements, and a miscellaneous category that defies neat categorization.

Cell culture medium may be optionally supplemented with supplementary components. The term “supplementary components” as used herein refers to components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source. In some embodiments, supplementary components may be added to the initial cell culture. In some embodiments, supplementary components may be added after the beginning of the cell culture.

Typically, components which are trace elements refer to a variety of inorganic salts included at micromolar or lower levels. For example, commonly included trace elements are zinc, selenium, copper, and others. In some embodiments, iron (ferrous or ferric salts) can be included as a trace element in the initial cell culture medium at micromolar concentrations. Manganese is also frequently included among the trace elements as a divalent cation (MnCl₂ or MnSO₄) in a range of nanomolar to micromolar concentrations. Numerous less common trace elements are usually added at nanomolar concentrations.

In some embodiments, methods and compositions provided herein involve cell cultures and cell culture media. The terms “culture” and “cell culture” as used herein refer to a cell population that is in a medium under conditions suitable to survival and/or growth of the cell population. As will be clear to those of ordinary skill in the art, in some embodiments, these terms as used herein refer to the combination comprising the cell population and the medium in which the population is present. In some embodiments, the cells of the cell culture comprise mammalian cells. In some embodiments, a cell culture comprises cells in suspension. In some embodiments, a cell culture comprises cells grown on a substrate.

In some embodiments, host cells provided herein which contain a recombinant nucleic acid construct provided herein may be used to produce a protein encoded by a nucleotide sequence of interest. Similarly, as provided herein, methods and compositions provided herein may be used to obtain host cells that contain a nucleotide sequence of interest, and polypeptides encoded by such nucleotide sequences of interest may be produced and purified. In addition, such host cells may be generated and cultured.

The present invention may be used with any cell culture method that is amenable to the desired process (e.g., introduction of a recombinant nucleic acid construct according to methods provided herein and production of a recombinant protein (e.g., an antibody)). As a non-limiting example, cells may be grown in batch or fed-batch cultures, where the culture is terminated after sufficient expression of the recombinant protein (e.g., antibody), after which the expressed protein (e.g., antibody) is harvested. Alternatively, as another non-limiting example, cells may be grown in batch-refeed, where the culture is not terminated and new nutrients and other components are periodically or continuously added to the culture, during which the expressed recombinant protein (e.g., antibody) is harvested periodically or continuously. Other suitable methods (e.g., spin-tube cultures) are known in the art and can be used to practice the present invention.

In some embodiments, provided herein are compositions containing polypeptides produced from host cells and according to methods provided herein, and one or more pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

### Methods

In some aspects, provided herein are methods of using nucleic acids and compositions provided herein. For example, in some embodiments, methods are provided for obtaining a host cell containing a nucleotide sequence of interest, wherein the cysteine selection marker system disclosed herein is used to select for cells that have obtained the nucleotide sequence of interest. As described elsewhere herein, this may be accomplished, for example, by coupling the nucleotide sequence of interest to one or both of the CBS and CTH genes in a nucleic acid construct; cells that have received the nucleic acid construct(s) containing the CBS and CTH genes may be selected for based on their ability to grown in cysteine-deficient media. In some embodiments, the CBS and CTH genes are included together in the same nucleic acid construct; with this format, a host cell only needs to receive a single construct to be converted from a cysteine auxotroph to a cysteine prototroph (because both the CBS and CTH genes enter the host cell on the same construct). In some other embodiments, the CBS and CTH genes are present on different nucleic acid constructs; with this format, a host cell needs to receive both constructs to be converted from a cysteine auxotroph to a cysteine prototroph. While the format of including the CBS and CTH genes on different nucleic acid constructs may increase the difficulty of obtaining a host cell that is converted from a cysteine auxotroph to a cysteine prototroph, it is useful, for example, where is desirable to introduce a first and a second nucleotide sequence of interest into the cell. By coupling the first nucleotide sequence of interest to the CBS gene and the second nucleotide sequence of interest to the CTH gene in two different nucleic acid constructs, cells that have obtained both the first and second nucleotide sequence of interest can be selected for based on cysteine prototrophy.

In some embodiments, the compositions and methods provided herein may be used in combination with one or more other selection marker systems, such that, for example, multiple different exogenous nucleic acids containing different selection markers can be introduced into a cell, and cells that receive all of the different exogenous nucleic acids of interest can be selected. Other selection marker systems that may be used in conjunction with the cysteine selection marker system disclosed herein include, for example, the glutamine synthetase (“GS”) selection marker, the hygromycin selection marker, the puromycin selection marker, the neomycin phosphortransferase (NPTII) selection marker, or the thymidine kinase selection marker.

As used herein, the term “selection marker gene” refers to a nucleotide sequence, in particular a gene encoding a polypeptide, under regulatory and functional control at least one regulatory element, in particular a promoter, wherein the gene encodes a polypeptide that allows for selection of host cells that express that polypeptide, alone or in combination with one or more additional polypeptides. For instance, in the context of the cysteine selection marker system provided herein, both CBS and CTH may be considered “selection marker genes”.

The GS marker system involves the GS gene. In the absence of glutamine in the growth medium, the glutamine synthetase (GS) activity is essential for the survival of mammalian cells in culture. Some mammalian cell lines, such as mouse myeloma lines, do not express sufficient GS to survive without added glutamine. With these cell lines a transfected GS marker gene can function as a selectable marker by permitting growth in a glutamine-free medium. Other cell lines, such as Chinese hamster ovary cell lines, express sufficient GS to survive without exogenous glutamine. In these cases, the GS inhibitor methionine sulfoximine (MSX) can be used to inhibit endogenous GS activity such that only transfectants with additional GS activity can survive.

Thus, for example, in some embodiments, provided herein is a method of obtaining a host cell containing a first exogenous nucleotide sequence of interest and a second exogenous nucleotide sequence of interest, the method comprising a) introducing into a population of cells i) a first nucleic acid construct comprising the first nucleotide sequence of interest, a CBS and a CTH gene and ii) a second nucleic acid construct comprising the second nucleotide sequence of interest and a selection marker selected from group consisting of glutamine synthetase (“GS”) selection marker, the hygromycin selection marker, the puromycin selection marker or the thymidine kinase selection marker, and b) selecting from the population of cells a host cell containing the first nucleic acid construct and the second nucleic acid construct, wherein the host cell is selected for both i) cysteine prototrophy and ii) the survival characteristic conferred by the respective GS selection marker, hygromycin selection marker, puromycin selection marker or thymidine kinase selection marker provided in the second nucleic acid construct. In another embodiment, provided herein is a method of obtaining a host cell containing a first exogenous nucleotide sequence of interest, a second exogenous nucleotide sequence of interest, and a third exogenous nucleotide sequence of interest, the method comprising a) introducing into a population of cells i) a first nucleic acid construct comprising the first nucleotide sequence of interest and a CBS gene, ii) a second nucleic acid construct comprising the second nucleotide sequence of interest and a CTH gene, and iii) a third nucleic acid construct comprising the third nucleotide sequence of interest and a selection marker selected from group consisting of glutamine synthetase (“GS”) selection marker, the hygromycin selection marker, the puromycin selection marker or the thymidine kinase selection marker, and b) selecting from the population of cells a host cell containing the first nucleic acid construct, the second nucleic acid construct, and the third nucleic acid construct wherein the host cell is selected for both i) cysteine prototrophy and ii) the survival characteristic conferred by the respective GS selection marker, hygromycin selection marker, puromycin selection marker or thymidine kinase selection marker provided in the third nucleic acid construct. In some embodiments, any of the methods described above may be used with the SSI or random integration approaches described herein.

In some embodiments, also provided herein are host cells which have been genetically engineered to overexpress one or more additional genes in the cysteine metabolism pathway, in addition to CBS and CTH. For example, some embodiments, provided herein is a host cell containing one or more nucleic acid constructs provided herein containing the CBS and CTH genes, wherein the host cell further comprises an exogenous copy of one or more genes selected from the group consisting of BHMT (betaine-homocysteine methyltransferase); BHMT2 (betaine-homocysteine methyltransferase 2); MTR (5-methyltetrahydrofolate-homocysteine methyltransferase); AHCY (S-adenosylhomocysteine hydrolase); MAT1A (methionine adenosyltransferase I, alpha); MAT2A (methionine adenosyltransferase II, alpha); MAT2B (methionine adenosyltransferase II, beta); DNMT1 (DNA methyltransferase (cytosine-5) 1); DNMT3A (DNA methyltransferase 3A); and DNMT3B (DNA methyltransferase 3B).

In some embodiments, such cells have improved cysteine metabolism and/or reduced production of undesirable metabolites. Also provided herein is a host cell containing one or more nucleic acid constructs provided herein containing the CBS and CTH genes, wherein the host cell has additionally been genetically modified to increase the endogenous gene expression of one or more of the above listed genes.

In some embodiments, compositions and methods provided herein may be used in combination with compositions and methods disclosed in PCT/IB2016/055666, which is hereby incorporated by reference for all purposes.

### Kits

In some embodiments, also provided herein are kits comprising one or more of the recombinant nucleic acid constructs, vectors, host cells, polypeptides, or media provided herein. For example, in some embodiments, provided herein is a kit comprising A) a recombinant nucleic acid construct comprising i) a nucleotide sequence of interest, and ii) a CBS gene, and B) a recombinant nucleic acid construct comprising i) a nucleotide sequence of interest, and ii) a CTH gene. In some embodiments, provided herein is a kit comprising A) a recombinant nucleic acid construct comprising a CBS gene, and B) a recombinant nucleic acid construct comprising a CTH gene. Optionally, components of a kit are provided in different containers (i.e. a first container and a second container) in the kit. Containers include, for example, vials, bottles, jars, flexible packaging (e.g., sealed Mylar or plastic bags), and the like. Optionally, a kit contains instructions for use of items in the kit in accordance with any of the methods of the invention described herein. Kits may optionally provide additional components such as buffers and interpretive information.

Exemplary embodiments (E) of the invention provided herein include:
E1. A recombinant nucleic acid construct comprising i) a nucleotide sequence of interest; ii) a cystathionine beta-synthase (CBS) gene; and iii) a cystathionase (cystathionine gamma-lyase) (CTH) gene.
E2. A recombinant nucleic acid construct comprising i) a nucleotide sequence of interest; and ii) a CBS gene.
E3. A recombinant nucleic acid construct comprising i) a nucleotide sequence of interest; and ii) a CTH gene.
E4. The recombinant nucleic acid construct of E1, wherein the nucleic acid construct further comprises a recombination target sequence.
E5. The recombinant nucleic acid construct of E2, wherein the nucleic acid construct further comprises a recombination target sequence.
E6. The recombinant nucleic acid construct of E3, wherein the nucleic acid construct further comprises a recombination target sequence.
E7. The recombinant nucleic acid construct of any of E4-E6, wherein the recombination target sequence is a FLP Recognition Target (“FRT”), lox, or Bxb1 sequence.
E8. The recombinant nucleic acid construct of any of E1-E7, wherein the nucleotide sequence of interest encodes a polypeptide of interest or an RNA molecule of interest.
E9. The recombinant nucleic acid construct of any of E1-E8, wherein the nucleotide sequence of interest is a first nucleotide sequence of interest, and wherein the recombinant nucleic acid construct further comprises a second nucleotide sequence of interest.
E10. The recombinant nucleic acid construct of E9, wherein the first nucleotide sequence of interest and the second nucleotide sequence of interest are transcribed as a single bicistronic mRNA transcript.
E11. The recombinant nucleic acid construct of E10, wherein the first nucleotide sequence of interest and second nucleotide sequence of interest are separately translated from the single bicistronic mRNA transcript into a first polypeptide and second polypeptide.
E12. The recombinant nucleic acid construct of any of E9-E11, wherein the first nucleotide sequence of interest encodes a first polypeptide comprising an antibody variable light (VL) region and wherein the second nucleotide sequence of interest encodes a second polypeptide comprising an antibody variable heavy (VH) region.
E13. A vector comprising the recombinant nucleic acid construct of any of E1-E12.
E14. The vector of E13, wherein the vector is a plasmid vector.
E15. The vector of E13, wherein the vector is a viral vector.
E16. The vector of any of E13-E15, wherein the vector further comprises a selection marker selected from the group consisting of an antibiotic selection marker, a glutamine synthetase selection marker, a hygromycin selection marker, a puromycin selection marker and a thymidine kinase selection marker.
E17. A host cell comprising any one or more of: the recombinant nucleic acid construct of any of E1-E12; the vector of any of E13-E16; or an exogenous CBS gene, an exogenous CTH gene, an exogenous GNMT gene, and optionally reduced expression or activity of the MTR gene or protein.
E18. A host cell comprising the recombinant nucleic acid construct of any of E1-E12, wherein the recombinant nucleic acid construct is stably integrated into a chromosome of the host cell.
E19. A host cell comprising the recombinant nucleic acid construct of E2 and the recombinant nucleic acid construct of E3, wherein the nucleotide sequence of interest of E2 is a first nucleotide sequence of interest and wherein the nucleotide sequence of interest of E3 is a second nucleotide sequence of interest.
E20. The host cell of E19, wherein at least one of the recombinant nucleic acid construct of E2 and the recombinant nucleic acid construct of E3 is stably integrated into a first chromosome of the host cell.
E21. The host cell of E20, wherein both the recombinant nucleic acid construct of E2 and the recombinant nucleic acid construct of E3 are stably integrated into the first chromosome of the host cell.
E22. The host cell of E20, wherein the recombinant nucleic acid construct of E2 is stably integrated into the first chromosome of the host cell and the recombinant nucleic acid construct of E3 is stably integrated into a second chromosome of the host cell.
E23. The host cell of any of E19-E22, wherein the first nucleotide sequence of interest encodes a first polypeptide comprising an antibody VH region and wherein the second nucleotide sequence of interest encodes a second polypeptide comprising an antibody VL region.
E24. The host cell of any of E17-E23, wherein the host cell is a mammalian cell.
E25. The host cell of E24, wherein the mammalian cell is a mouse cell, a human cell, or a CHO cell.
E26. Use of a host cell of any of E17-E25 for production of a polypeptide or RNA molecule encoded by the nucleotide sequence of interest.
E27. Use of a host cell of any of E19-E25 for production of a first polypeptide or first RNA molecule encoded by the first nucleotide sequence of interest and for production of a second polypeptide or second RNA molecule encoded by the second nucleotide sequence of interest.
E28. A recombinant polypeptide produced by the host cell of any of E17-E25.
E29. A composition comprising the recombinant nucleic acid of E2 and the recombinant nucleic acid of E3.
E30. A composition comprising a first vector comprising the recombinant nucleic acid of E2 and a second vector comprising the recombinant nucleic acid of E3, wherein the nucleotide sequence of interest of E2 is a first nucleotide sequence of interest and wherein the nucleotide sequence of interest of E3 is a second nucleotide sequence of interest.
E31. A composition comprising a recombinant polypeptide of E28 and a pharmaceutically acceptable excipient.
E32. A composition comprising a host cell of any of E17-E25, and a cell culture medium.
E33. A composition comprising a host cell, a recombinant nucleic acid construct of any of E1-E12, and a cell culture medium.
E34. The composition of E33, wherein the host cell comprises a chromosome comprising a landing pad, wherein the landing pad comprises a recombination target site.
E35. A composition comprising a host cell, a recombinant nucleic acid construct of E2, a recombinant nucleic acid construct of E3, and a cell culture medium, wherein the nucleotide sequence of interest of E2 is a first nucleotide sequence of interest and wherein the nucleotide sequence of interest of E3 is a second nucleotide sequence of interest.
E36. The composition of E35, wherein the host cell comprises a first landing pad and a second landing pad, wherein the first landing pad comprises a first recombination target site and the second landing pad comprises a second recombination target site.
E37. The composition of E36, wherein the host cell comprises a first chromosome comprising the first landing pad and a second chromosome comprising the second landing pad.
E38. The composition of any of E32-E37, wherein the cell culture medium is cysteine-deficient.
E39. The composition of E38, wherein the cell culture medium comprises less than 2 mM cysteine.
E40. The composition of E39, wherein the cell culture medium comprises less than 500 μM cysteine.
E41. A method of obtaining a host cell comprising an exogenous nucleotide sequence of interest, the method comprising:
   a) exposing a population of cells to an exogenous nucleic acid construct comprising the nucleotide sequence of interest, wherein the exogenous nucleic acid construct further comprises: i) a CBS gene, and ii) a CTH gene;
   b) culturing the population of cells exposed to the exogenous nucleic acid construct in a cysteine-deficient medium; and
   c) obtaining from the population of cells exposed to the exogenous nucleic acid construct a host cell comprising the exogenous nucleotide sequence of interest, wherein the host cell comprising the exogenous nucleotide sequence of interest comprises the exogenous nucleic acid construct, and wherein the host cell comprising the exogenous nucleotide sequence of interest has a greater ability to proliferate in a cysteine-deficient cell culture medium than a corresponding cell that does not contain the exogenous nucleic acid construct.
E42. The method of E41, wherein the exogenous nucleic acid construct further comprises a recombination target sequence.
E43. The method of E42, wherein a chromosome of the host cell comprises a first landing pad, wherein the first landing pad comprises a recombination target site.
E44. The method of E43, wherein the nucleic acid construct recombination target sequence and the chromosomal recombination target site are FLP, lox, or Bxb1 sequences.
E45. A method of obtaining a cell comprising a first exogenous nucleotide sequence of interest and a second exogenous nucleotide sequence of interest, the method comprising:
   a) exposing a population of cells to I) a first exogenous nucleic acid construct comprising i) the first exogenous nucleotide sequence of interest and ii) a CBS gene, and II) a second exogenous nucleic acid construct comprising i) the second exogenous nucleotide sequence of interest and ii) a CTH gene; and
   b) culturing the population of cells exposed to the first exogenous nucleic acid construct and the second exogenous nucleic acid construct in a cysteine-deficient medium; and
   c) obtaining from the population of cells exposed to the first exogenous nucleic acid construct and the second exogenous nucleic acid construct a host cell comprising the first exogenous nucleotide sequence of interest and the second exogenous nucleotide sequence of interest, wherein the host cell comprising the first exogenous nucleotide sequence of interest and the second exogenous nucleotide sequence of interest comprises the first exogenous nucleic acid construct and the second exogenous nucleic acid construct, and wherein the host cell comprising the first exogenous nucleic acid construct and the second exogenous nucleic acid construct has a greater ability to proliferate in a cysteine-deficient cell culture medium than a corresponding cell that does not contain the first exogenous nucleic acid construct and the second exogenous nucleic acid.
E46. The method of E45, wherein the first exogenous nucleic acid construct further comprises a recombination target sequence.
E47. The method of E45, wherein the second exogenous nucleic acid construct further comprises a recombination target sequence.
E48. The method of E45, wherein the first exogenous nucleic acid construct further comprises a first recombination target sequence, and wherein the second exogenous nucleic acid construct further comprises a second recombination target sequence.
E49. The method of any of E46-E48, wherein a chromosome of the host cell comprises a first landing pad and a second landing pad, wherein the first landing pad comprises a first recombination target site and the second landing pad comprises a second recombination target site.
E50. The method of any of E46-E48, wherein a first chromosome of the host cell comprises a first landing pad, wherein the first landing pad comprises a first recombination target site, and wherein a second chromosome of the host cell comprises a second landing pad, wherein the second landing pad comprises a second recombination target site.
E51. The method of any of E49-E50, wherein the nucleic acid construct recombination target sequences and the chromosomal recombination target sites comprise FLP, lox, or Bxb1 sequences.
E52. A method of producing a host cell comprising an exogenous nucleotide sequence of interest, the method comprising:
   a) introducing into a host cell an exogenous nucleic acid construct comprising the nucleotide sequence of interest, wherein the exogenous nucleic acid construct further comprises: i) a CBS gene, and ii) a CTH gene;
   b) culturing the host cell comprising the exogenous nucleic acid construct in a cysteine-deficient medium, wherein the host cell comprising the exogenous nucleic acid construct proliferates more rapidly in the cysteine-deficient medium than a corresponding otherwise identical host cell that lacks the exogenous nucleic acid construct.
E53. The method of E52, wherein the exogenous nucleic acid construct is stably integrated into a chromosome of the host cell.
E54. The method of E53, wherein the exogenous nucleic acid construct is stably integrated into the chromosome by homologous recombination between the exogenous nucleic acid construct and the chromosome.
E55. The method of E54, wherein the integration of the exogenous nucleic acid construct into the chromosome is facilitated by a viral vector or an exogenous nuclease.
E56. A method of producing a host cell comprising a first exogenous nucleotide sequence of interest and a second exogenous nucleotide sequence of interest, the method comprising:
   a) introducing into a host cell I) a first exogenous nucleic acid construct comprising i) the first exogenous nucleotide sequence of interest and ii) a CBS gene and II) a second exogenous nucleic acid construct comprising i) the second exogenous nucleotide sequence of interest and ii) a CTH gene; and
   b) culturing the host cell comprising the first exogenous nucleic acid construct and the second exogenous nucleic acid construct in a cysteine-deficient medium, wherein the host cell comprising the first exogenous nucleic acid construct and the second exogenous nucleic acid construct proliferates more rapidly in the cysteine-deficient medium than a corresponding otherwise identical host cell that lacks the first exogenous nucleic acid construct and second exogenous nucleic acid construct.
E57. The method of E56, wherein the first exogenous nucleic acid construct and the second exogenous nucleic acid construct are both stably integrated into a first chromosome of the host cell, or wherein the first exogenous nucleic acid construct is stably integrated into a first chromosome of the host cell and the second exogenous nucleic acid construct is stably integrated into a second chromosome of the host cell.
E58. The method of E57, wherein the first exogenous nucleic acid construct and the second exogenous nucleic acid construct are stably integrated into the chromosome by homologous recombination between the respective exogenous nucleic acid construct and the chromosome.
E59. The method of E58, wherein the integration of the exogenous nucleic acid constructs is facilitated by a viral vector or an exogenous nuclease.
E60. The method E55 or E59, wherein the viral vector is an adeno-associated virus vector that mediates homologous recombination.
E61. The method of any of E41-E60, wherein the cysteine deficient medium comprises less than 2 mM cysteine.
E62. The method of any of E41-E61, wherein the cysteine deficient medium comprises less than 500 μM cysteine.
E63. The recombinant nucleic construct, vector, host cell, composition, or method of any of the above embodiments, wherein the CBS gene encodes a polypeptide comprising the amino acid sequence shown in SEQ ID NO: 1, or a sequence with at least 90% homology thereof.
E64. The recombinant nucleic construct, vector, host cell, composition, or method of any of the above embodiments, wherein the CBS gene comprises a DNA sequence shown in SEQ ID NO: 2, or a sequence with at least 90% homology thereof.
E65. The recombinant nucleic construct, vector, host cell, composition, or method of any of the above embodiments, wherein the CTH gene encodes a polypeptide comprising the amino acid sequence shown in SEQ ID NO: 3, or a sequence with at least 90% homology thereof.
E66. The recombinant nucleic construct, vector, host cell, composition, or method of any of the above embodiments, wherein the CTH gene comprises a DNA sequence shown in SEQ ID NO: 4, or a sequence with at least 90% homology thereof.
E67. A method of obtaining a host cell that is a cysteine prototroph, the method comprising increasing the expression of the genes CBS, CTH, and GNMT in the cell.
E68. The method of E67 further comprising reducing the expression or activity of the MTR gene or protein.
E69. The method of E67 or E68, wherein increasing the expression of the genes comprises introducing exogenous copies of the genes into the host cell.
E70. The method of E69, wherein reducing the expression or activity of the MTR gene or protein comprising inhibiting the MTR protein with a small molecule inhibitor.
E71. The method of any of E67-E70, wherein the host cell is a CHO cell.
E72. A host cell comprising an exogenous cystathionine beta-synthase (CBS) gene and an exogenous cystathionase (cystathionine gamma-lyase) (CTH) gene.
E73. The host cell of E72, further comprising an exogenous glycine N-methyltransferase (GNMT) gene.
E74. The host cell of E72 or E73, wherein at least one of the exogenous CBS gene and exogenous CTH gene is stably integrated into a chromosome of the host cell.
E75. The host cell of E73, wherein the exogenous CBS gene, exogenous CTH gene, and exogenous GNMT gene are each stably integrated into a chromosome of the host cell.
E76. The host cell of any of E72-E75, wherein the host cell is a mammalian cell.
E77. The host cell of E76, wherein the mammalian cell is a mouse cell, a human cell, or a Chinese Hamster Ovary (CHO) cell.
E78. Use of a host cell of any of E72-E77 for production of a recombinant polypeptide.
E79. A recombinant polypeptide produced by the host cell of any of E72-E77.
E80. The use of E78 or the recombinant polypeptide of E79, wherein the recombinant polypeptide is polypeptide of a monoclonal antibody.
E81. A composition comprising a host cell of any one of E72-E77, and a cell culture medium.
E82. The composition of E81, wherein the cell culture medium is cysteine-deficient.
E83. The composition of E82, wherein the cell culture medium comprises less than 2 mM cysteine, less than 1 mM cysteine, less than 500 μM cysteine, less than 200 μM cysteine, less than 100 μM cysteine, less than 50 μM cysteine, less than 10 μM cysteine, or 0 μM cysteine.
E84. The composition of any one of E81-E83, wherein the medium is homocysteine-deficient.
E85. The composition of E84, wherein the cell culture medium comprises less than 2 mM homocysteine, less than 1 mM homocysteine, less than 500 μM homocysteine, less than 200 μM homocysteine, less than 100 μM homocysteine, less than 50 μM homocysteine, less than 10 μM homocysteine, or 0 μM homocysteine.
E86. A method of obtaining a host cell having improved growth characteristics in cysteine-deficient media, the method comprising increasing the expression of the genes CBS and CTH in the host cell, wherein the host cell has improved growth characteristics as compared to an otherwise identical cell that does not have the increased expression of CBS and CTH in the cell.
E87. The method of E86, further comprising increasing the expression of the gene GNMT in the host cell, wherein the host cell has improved growth characteristics as compared to an otherwise identical cell that does not have the increased expression of CBS, CTH, and GNMT in the cell.
E88. The method of E86 or E87, wherein increasing the expression of the genes comprises introducing exogenous copies of the respective genes into the host cell.
E89. The method of any one of E86-E88 further comprising reducing the expression or activity of the methionine synthase (MTR) gene or protein in the cell.
E90. The method of E89, wherein reducing the expression or activity of the MTR gene or protein comprising inhibiting the MTR protein with a small molecule inhibitor.
E91. The method of E90, wherein the small molecule inhibitor is sodium nitroprusside (SNP) E92. The method of any of E86-E91, wherein the host cell is a mammalian cell.
E93. The method of E92, wherein the mammalian cell is a mouse cell, a human cell, or a Chinese Hamster Ovary (CHO) cell.
E94. The host cell, use, recombinant polypeptide, composition or method of any one of E72-E93, wherein any one or more of:
   a) the CBS gene encodes a polypeptide comprising the amino acid sequence shown in SEQ ID NO: 1, or a sequence with at least 90% homology thereof;
   b) the CBS gene comprises a DNA sequence shown in SEQ ID NO: 2, or a sequence with at least 90% homology thereof;
   c) the CTH gene encodes a polypeptide comprising the amino acid sequence shown in SEQ ID NO: 3, or a sequence with at least 90% homology thereof;
   d) the CTH gene comprises a DNA sequence shown in SEQ ID NO: 4, or a sequence with at least 90% homology thereof;
   e) the GNMT gene encodes a polypeptide comprising the amino acid sequence shown in SEQ ID NO: 5, or a sequence with at least 90% homology thereof; or
   f) the GNMT gene comprises a DNA sequence shown in SEQ ID NO: 6, or a sequence with at least 90% homology thereof.
E94. The composition or method of any one of E72-E93 or any of E1-E71 comprising a cell culture medium, wherein any one or more of:
   a) the cell culture medium contains added beta mercaptoethanol (BME);
   b) the cell culture medium does not contain added BME;
   c) the cell culture medium contains added oleic acid (OA);
   d) the cell culture medium does not contain added OA;
   e) the cell culture medium contains added ferroptosis inhibitors;
   f) the cell culture medium does not contain added ferroptosis inhibitors;
   g) the cell culture medium contains added ferroptosis inhibitors, wherein the ferroptosis inhibitor is ferrostatin 1 (Fer1) or vitamin K1; and
   h) the cell culture medium does not contain added ferroptosis inhibitors, wherein the ferroptosis inhibitor is ferrostatin 1 (Fer1) or vitamin K1.

Incorporated by reference herein for all purposes is the content of U.S. Provisional Patent Application No. 63/305,833 (filed Feb. 2, 2022) and U.S. Provisional Patent Application No. 63/479,059 (filed Jan. 9, 2023).

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1: Determination of the Expression Levels of Enzymes in the Methionine Cycle and the Transsulfuration Pathway in a CHO Cell Line to Identify Metabolic Engineering Targets that can Confer Cysteine Prototrophy

### Goal

This experiment was performed to determine the gene expression levels, and by extension, the enzymatic activity of enzymes that might be involved in conferring cysteine prototrophy in a CHO cell line which expresses an IgG antibody. Gene expression of enzymes in the methionine cycle and transsulfuration pathway was probed using a Real Time Quantitative Polymerase Chain Reaction (“RT-qPCR”) assay.

### Materials and Methods

RT-qPCR assay was used to assess relative gene expression levels of enzymes in the transsulfuration metabolic pathway. RT qPCR measures transcript abundance, and hence, gene expression by amplifying a target cDNA sequence using PCR in combination with a detection reagent (i.e. SYBR Green). SYBR green is a molecule that fluoresces when bound to double stranded DNA and the fluorescence can be measured in real time during the RT qPCR assay. The amount of fluorescence is directly proportional to the amount of double stranded PCR product (also called amplicon) in the reaction. Relative gene expression levels are determined by measuring the number of PCR cycles required for SYBR green fluorescence to surpass the background fluorescence and increase logarithmically. This cycle number is commonly referred to as the C_{T} (Threshold Cycle). A transcript in high abundance would have a lower C_{T} value as it would require fewer PCR cycles for the fluorescence to surpass the background fluorescence where, conversely, a transcript in lower abundance would have a higher C_{T} value as it would require more PCR cycles for the fluorescence to surpass the background level.

The RT qPCR assay was performed using an Applied Biosystems 7500 Real Time PCR system (Applied Biosystems) and the PowerUP SYBR Green Master Mix reagent (Life Technologies). The PCR primers were designed using the Primer3 algorithm based on genomic DNA sequences contained in the Chinese Hamster Ovary (CHO) genome browser (chogenome.org). RNA was prepared from the CHO cell line using the Qiagen RNeasy Kit (Qiagen) which was in turn used as template for oligo dT primed cDNA synthesis using the SuperScript III First-Strand Synthesis System for RT-PCR (Life Technologies). The C_{T} values of the targeted metabolic genes were tabulated and compared to the C_{T} value of a well characterized housekeeping gene, beta-Actin (B-Actin). The difference between the C_{T} of the target gene and the C_{T} of B-Actin was reported as the ΔC_{T}. High ΔC_{T} value indicates low gene expression level.

### Results

The C_{T} and ΔC_{T} values for the genes in the methionine cycle and the transsulfuration pathway for cell line A are shown in Table 1. The genes listed in Table 1 are as follows: B-Actin (Actin, beta); CTH (cystathionase (cystathionine gamma-lyase)); CBS (cystathionine beta-synthase); BHMT (betaine-homocysteine methyltransferase); BHMT2 (betaine-homocysteine methyltransferase 2); MTR (5-methyltetrahydrofolate-homocysteine methyltransferase); AHCY (S-adenosylhomocysteine hydrolase); MAT1A (methionine adenosyltransferase I, alpha); MAT2A (methionine adenosyltransferase II, alpha); MAT2B (methionine adenosyltransferase II, beta); DNMT1 (DNA methyltransferase (cytosine-5) 1); DNMT3A (DNA methyltransferase 3A); DNMT3B (DNA methyltransferase 3B).

The gene expression data from the methionine cycle and the transsulfuration pathway indicates that the CHO cell line has low expression of the CBS, CTH, BHMT2, and MAT1A genes (low gene expression defined as C_{T} value greater than 30 cycles).

The CBS and CTH genes are directly involved in cysteine biosynthesis from homocysteine, an intermediate of methionine cycle, as they encode the enzymes responsible for the stepwise conversion of homocysteine (and serine) to cystathionine and the conversion of cystathionine to cysteine, respectively.

Based on their low expression levels (evidenced by their respective high C_{T} values and high ΔC_{T}) and the biochemistry of the methionine cycle and the transsulfuration pathway, the genes CBS and CTH, were selected as possible genes for conferring cysteine prototrophy and for use in a cysteine selection marker system.

TABLE 1
Gene expression analysis of the methionine cycle and the transsulfuration
pathway genes in the CHO cell line using RT-qPCR assay

| RT qPCR | | | |
|---|---|---|---|
| Reaction | Gene | C_{T} | ΔC_{T} |
| 1 | B-Actin | 15.89 | |
| | CTH | 34.62 | 18.73 |
| | CBS | 32.96 | 17.07 |
| | BHMT2 | 31.18 | 15.29 |
| | MTR | 25.94 | 10.05 |
| | AHCY | 21.97 | 6.07 |
| | MAT1A | 33.80 | 17.90 |
| | MAT2B | 23.84 | 7.94 |
| | DNMT1 | 22.80 | 6.90 |
| | DNMT3A | 24.84 | 8.94 |
| 2 | B-Actin | 16.91 | |
| | MAT2A | 22.92 | 6.01 |

### Example 2: Experiment to Select CHO Cells Overexpressing Mouse Orthologs of CBS and CTH Genes Using Antibiotics as Selection Pressure

### Goal

The goal of this experiment was to generate cell lines that overexpress the mouse orthologs of CBS and CTH genes. As described above in Example 1, the genes CBS and CTH were selected as possible genes for conferring cysteine prototrophy in CHO cells based on their low expression levels and the biochemistry of the transsulfuration pathway.

Plasmid vectors with expression cassettes containing mouse orthologs of CBS and CTH were transfected into a CHO cell line which expresses an IgG antibody. The transfected cells were tested for selection and subsequent outgrowth in medium supplemented with antibiotics. The expression level of mouse orthologs of CBS and CTH genes were probed in the selected population of cells. Outgrowth in medium supplemented with selective antibiotics and concurrent higher expression levels of CBS and CTH genes in this cell pool established successful transgene integration, expression, and, by extension, enzymatic activity of the mouse orthologs of CBS and CTH.

### Materials and Methods

Expression vectors for CBS and CTH genes were constructed using mouse cDNA sequences from the MGC collection. The sequences were provided by GE Dharmacon as E. coli glycerol stocks containing shuttle vectors with cDNAs of the target genes. PCR primers were designed using the Primer3 algorithm to amplify the coding regions of the cDNAs in reactions with the proof-reading polymerase Pfu Turbo HotStart 2× Master Mix (Agilent). The PCR products were cloned into commercially available constitutive expression vectors with different antibiotic resistance genes to allow for individual selection of the expression plasmids. In addition, control vectors were also provided to serve as a negative control (transfection control). The vectors were sequence confirmed by WyzerBiosciences (Cambridge, MA). The expression and control plasmids were transfected into cell line B using the GenePulser XCell electroporator (BioRad) and recovered in the presence of antibiotic. Viable cell density and percent viability of transfected cells were monitored in the days following transfection.

### Results

Cells were transfected with expression vectors including the CTH expression vector, CBS and CTH expression vectors (2 separate vectors), control vector for CTH expression vector (control vector containing chloramphenicol acetyl transferase (CAT)), or control vectors for CBS and CTH expression vectors (empty control vector for CBS expression vector and control vector for CTH expression vector containing CAT gene).

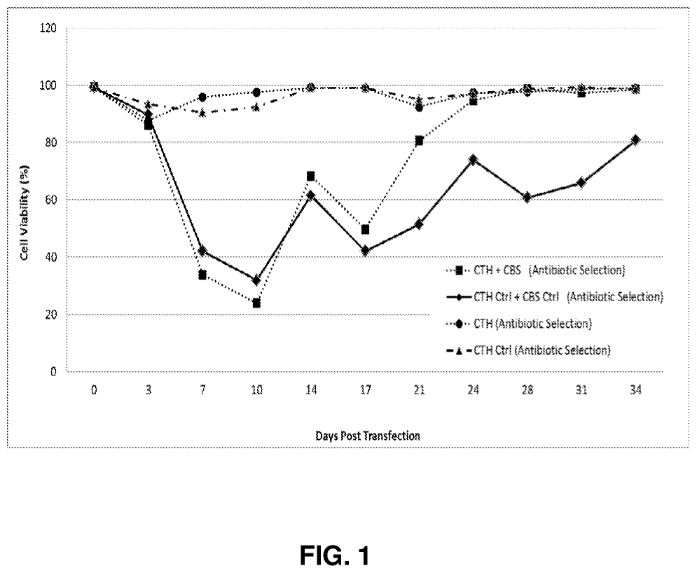

The transfected cell pools were selected in medium containing cysteine that was supplemented with selective antibiotic(s) (antibiotic resistance conferred by transfected plasmid). The description of transfection conditions and selection pressure used is listed in Table 2. Table 2 also summarizes the cell recovery results for different conditions from the experiment. FIG. 1 shows the recovery viability profiles of the transfected cells under antibiotic selection pressure. These cells also had high expression levels of mouse orthologs of CBS and CTH when compared to untransfected cell line B (Table 3).

TABLE 2
Summary of the CBS and CTH transfections performed,
selection pressures used, and cell recovery outcomes

| Cysteine | Transfection | Selective | |
|---|---|---|---|
| Supplementation | Treatment | Medium | Result |
| Cysteine | CTH Vector | (+) Cysteine, (+) | Cells |
| Supplemented | | Antibiotic | recovered |
| Medium | | | |
| Cysteine | CBS and CTH | (+) Cysteine, (+) | Cells |
| Supplemented | Vectors | Antibiotic | recovered |
| Medium | | | |
| Cysteine | CBS Control Vector | (+) Cysteine, (+) | Cells |
| Supplemented | (empty vector) | Antibiotic | recovered |
| Medium | | | |
| Cysteine | CTH Control Vector | (+) Cysteine, (+) | Cells |
| Supplemented | (CAT gene) | Antibiotic | recovered |
| Medium | | | |
| Cysteine | CBS Control Vector | (+) Cysteine, (+) | Cells |
| Supplemented | (empty vector) and | Antibiotic | recovered |
| Medium | CTH Control Vector | | |
| | (CAT gene) | | |

TABLE 3
RT qPCR analysis of expression levels of mouse orthologs
of CBS and CTH in the cells transfected with both
the genes and selected using antibiotics

| Condition | Gene | C_{T} | ΔC_{T} |
|---|---|---|---|
| Wild Type Cells | B-Actin | 15.86 | |
| | Mouse CTH | 36.96 | 21.10 |
| | Mouse CBS | 28.08 | 12.22 |
| Cells Transfected with | B-Actin | 16.64 | |
| CTH and CBS Vectors and | Mouse CTH | 20.43 | 3.79 |
| Selected in Antibiotics | Mouse CBS | 20.74 | 4.10 |
| Cells Transfected with | B-Actin | 15.93 | |
| Control Vectors for CTH | Mouse CTH | 32.95 | 17.02 |
| and CBS and Selected in | Mouse CBS | 26.98 | 11.06 |
| Antibiotics | | | |

### Example 3: Experiment to Demonstrate Use of CBS and CTH Genes as Cysteine Prototrophic Selection Pressure for Selecting Transfected Cells

The goal of this experiment is to build expression vectors that each contain one or more nucleotide sequences of interest and that together confer cysteine prototrophy, such that cells containing both expression vectors can be easily selected via a cysteine-based selection marker system.

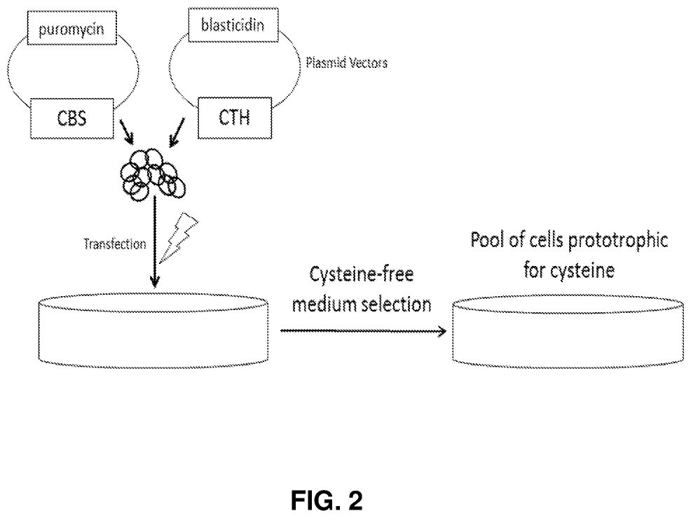

Two expression vectors are prepared, where each vector contains the mouse ortholog of either the CBS or the CTH gene (FIG. 2). Thus, the vectors contain, respectively, an expression cassette containing either A) the CBS gene or B) the CTH gene. The host cells are transfected with the aforementioned vectors simultaneously (FIG. 2) and the transfected cells are selected using growth/selection medium lacking cysteine (and cystine). The selected population of cells express the CBS and CTH genes, thus demonstrating requirement of CBS and CTH for cysteine prototrophy.

### Example 4: Experiment to Demonstrate Use of CBS and CTH Genes as Cysteine Prototrophic Selection Pressure for Selecting Cells Containing One or More Exogenous Nucleotide Sequence(s) of Interest, Using a Cell Line with a Single Landing Pad

The goal of this experiment is to build an expression vector that contains one or more nucleotide sequences of interest and confers cysteine prototrophy, such that cells containing the expression vector can be easily selected via a cysteine deficient medium-based selection system. The expression vector described in this Example may be used with, for example, a site-specific integration (SSI) cell line that contains a single landing pad.

Since both CBS and CTH expression are required for cysteine prototrophy, the expression vector contains the mouse orthologs of both the genes. The vector employs an IRES element such that both genes are expressed as a single bicistronic transcript using the same promotor (and promoter-upstream) element(s). However, the proteins are translated separately from the RNA segment for each gene. (The IRES element refers to an “internal ribosome entry site”; an IRES element supports translation initiation). As the order of the genes (CBS and CTH) separated by an IRES element can influence the levels of proteins translated from the RNA segment, two different versions of the vector are constructed. In one case the order of vector DNA element containing CBS and CTH genes is promoter-CBS-IRES-CTH and the order in the other case is promoter-CTH-IRES-CBS. Both versions of the vector also contain a first nucleotide sequence of interest and a second nucleotide sequence of interest, in which the first nucleotide sequence of interest encodes the heavy chain of an IgG molecule and the second nucleotide sequence of interest encodes the light chain of an IgG molecule. Thus, both versions of the vector contain an expression cassette containing i) the CBS gene, ii) the CTH gene, iii) a first nucleotide sequence of interest (encoding the heavy chain for IgG), and iv) a second nucleotide sequence of interest (encoding the light chain for IgG). The expression cassette is flanked by recombination target sequences that correspond to a recombination target site in the landing pad of the host cell.

The host cell with a single landing pad is transfected with one of the two aforementioned versions of the vector. The transfected cells are selected using growth/selection medium lacking cysteine. Successful transfection and selection of host cells containing the expression cassette containing the CBS and CTH genes and the first and second nucleotide sequences of interest is due to occupancy of the landing pad by the expression cassette containing the CBS and CTH genes and the first and second nucleotide sequences of interest. Expression of the CBS and CTH genes by a host cell which contains the expression cassette permits cell growth in the medium lacking cysteine (and containing homocysteine). In such cells, the first and second nucleotide sequences of interest (e.g. genes encoding the light and heavy chains of IgG) are also expressed from the expression cassette at the landing pad location.

While the above example is described in the context of a host cell containing a landing pad for site specific integration of the expression cassette, the method may also be performed in cells without a landing pad, in which cells that have undergone random integration of the expression cassette are selected.

### Example 5: Experiment to Demonstrate Use of CBS and CTH Enzymes as Cysteine Prototrophic Selection Pressure for Selecting Cells Containing One or More Exogenous Nucleotide Sequence(s) of Interest, Using a Cell Line with Two Landing Pads

The goal of this experiment is to build expression vectors that each contain one or more nucleotide sequences of interest and that together confer cysteine prototrophy, such that cells containing both expression vectors can be easily selected via a cysteine-based selection system. The expression vectors described in this Example may be used with, for example, a site-specific integration (SSI) cell line that contains two landing pads.

Since both CBS and CTH expression are required for cysteine prototrophy, two expression vectors are prepared, where each vector contains the mouse ortholog of either the CBS or the CTH gene, and at least one nucleotide sequence of interest. Thus, the vectors contain, respectively, an expression cassette containing either A) the CBS gene and at least a first nucleotide sequence of interest or B) the CTH gene and at least a second nucleotide sequence of interest. In this Example, in some instances, the first nucleotide sequence of interest encodes the heavy chain for an IgG molecule and the second nucleotide sequence of interest encodes the light chain for an IgG molecule. The expression cassettes of the respective vectors are flanked by recombination target sites that correspond to a recombination target site in the landing pad of the host cell. Optionally, the first landing pad and the second landing pad of the host cell contain different types/sequences for the recombination target site, and the expression cassettes in the respective vectors also contain different recombination target sites that correspond to the different landing pads of the host cell. Thus, for example, the expression cassette of the first vector (e.g. that contains the CBS gene and the first nucleotide sequence of interest) may be flanked by recombination target sequences that correspond to the recombination target site in the first landing pad of the host cell, and the expression cassette of the second vector (e.g. that contains the CTH gene and the second nucleotide sequence of interest) may be flanked by different recombination target sequences that correspond to the recombination target site in the second landing pad of the host cell. Use of different recombination target site sequences permits, for example, targeting of particular exogenous expression cassettes to particular landing pad locations in the host cell genome.

The host cell with the two landing pads is transfected with the aforementioned vectors simultaneously and the transfected cells are selected using growth/selection medium lacking cysteine (and cystine). This selection media can include L-homocysteine. Successful transfection and selection of host cells containing both the expression cassette containing the CBS gene and the first nucleotide sequence of interest and the expression cassette containing the CTH gene and the second nucleotide sequence of interest is due to occupancy of the first and second landing pads by the two different expression cassettes. Expression of the CBS and CTH genes in a host cell, which contains both expression cassettes, permits cell growth in the medium lacking cysteine. In such cells, the first and second nucleotide sequences of interest (e.g. genes encoding the light and heavy chains for IgG) are also expressed from the expression cassettes at the respective landing pad location.

While the above example is described in the context of a host cell containing a first and second landing pad for site specific integration of the expression cassette, the method may also be performed in cells without landing pads, in which cells that have undergone random integration of the both the CBS-containing expression cassette and the CTH-containing expression cassette are selected.

Also, while the above example is described in the context of the vectors containing, respectively, an expression cassette containing either A) the CBS gene and at least a first nucleotide sequence of interest or B) the CTH gene and at least a second nucleotide sequence of interest, wherein the first nucleotide sequence interest encodes the heavy chain for an IgG molecule and the second nucleotide sequence of interest encodes the light chain for an IgG molecule, other embodiments are also contemplated. For example, in some instances, the expression cassettes may contain either A) the CBS gene and at least a first nucleotide sequence of interest and a second nucleotide sequence of interest, or B) the CTH gene and at least a first nucleotide sequence of interest and a second nucleotide sequence of interest, wherein the first nucleotide sequence interest encodes the heavy chain for an IgG molecule and the second nucleotide sequence of interest encodes the light chain for an IgG molecule. Thus, in this instance, introduction of the CBS gene-containing and CTH gene-containing expression cassettes into a cell introduces 2 copies of each of both the IgG heavy chain and IgG light chain-encoding genes. Introduction of these cassettes increases the number of genes encoding the IgG heavy chain and IgG light chain molecules and may result in increased protein produced from the genes, as compared to host cells containing single copies of the genes.

### Example 6: Experiment to Demonstrate Combination of CBS, CTH, and GNMT Genes for Cysteine Prototrophic Selection

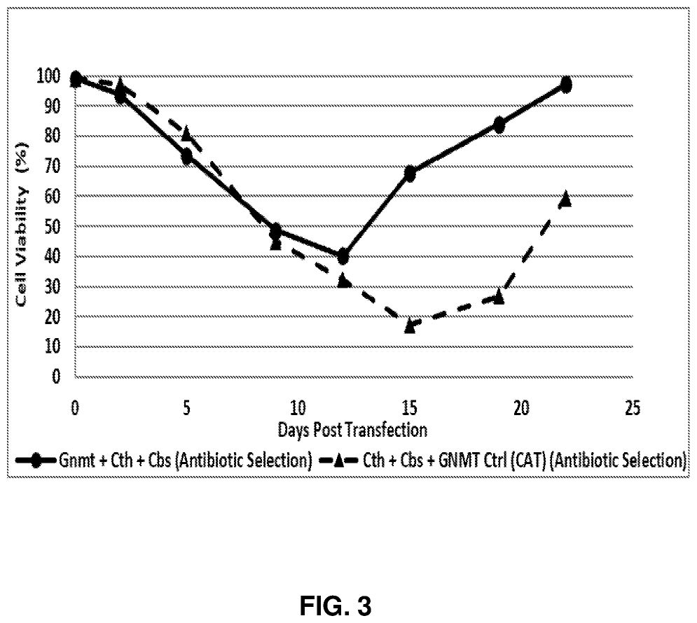

CHO cell pools overexpressing CBS and CTH and genes encoding a monoclonal antibody (mAb) were transfected with a vector containing either mouse glycine N-methyltransferase (GNMT) gene or a control vector (containing CAT gene). Table 4 lists the two conditions. The pools were recovered in antibiotic selection using CD CHO medium containing cysteine (FIG. 3).

TABLE 4
Summary of the GNMT transfections performed on cells
previously transfected with CTH and CBS vectors,
selection pressures used, cell recovery outcomes

| Cysteine | Transfection | Selective | |
|---|---|---|---|
| Supplementation | Treatment | Medium | Result |
| Cysteine | GNMT Vector | (+) Cysteine; (+) | Cells |
| Supplemented | | Antibiotics | Recovered |
| Medium | Control Vector (CAT | (+) Cysteine; (+) | Cells |
| | gene) | Antibiotics | Recovered |

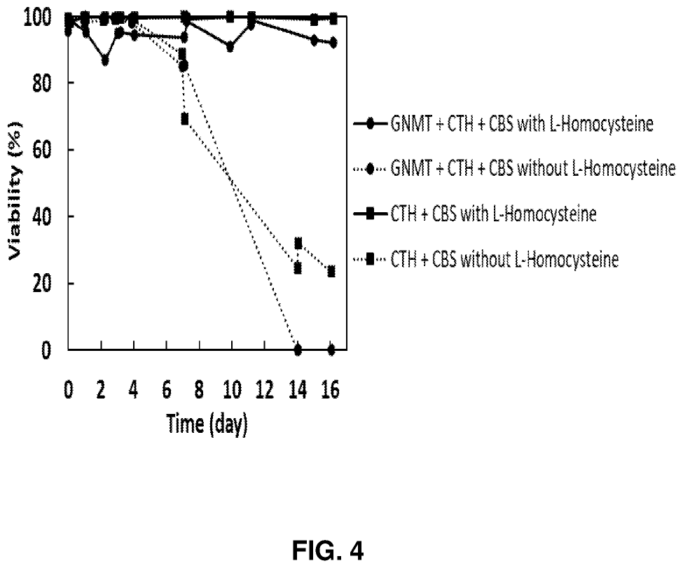

Next, the growth of CHO cell pools overexpressing CBS and CTH (referred to as “CBS+CTH” cells) or overexpressing CBS, CTH and GNMT (referred to as “GNMT” cells) was evaluated in cysteine free CD CHO medium or L-homocysteine supplemented cysteine free CD CHO medium. Both these pools lost viability in cysteine free conditions but exhibited growth in L-homocysteine supplemented conditions (FIG. 4). Subsequently, as described in more detail in Example 7, the CBS+CTH and the GNMT cell pools were adapted to grow in L-homocysteine free conditions. For both the CBS+CTH and GNMT cell pools, gene expression levels of CTH, CBS, GNMT and MTR were measured before and after adaptation to L-homocysteine free conditions (Table 5).

TABLE 5
RT qPCR analysis of expression levels of mouse orthologs of CBS,
CTH and GNMT in a) the cells transfected with the CTH and CBS genes
and selected using antibiotics, with and without adaptation to L-
Homocysteine depleted conditions and b) the cells transfected with
the CTH, CBS and GNMT genes and selected using antibiotics, with
and without adaptation to L-Homocysteine depleted conditions

| Condition | Gene | Ct | ΔCt |
|---|---|---|---|
| Wild Type Cells | β-Actin | 15.89 | |
| | Mouse CTH | 34.62 | 18.73 |
| | Mouse CBS | 32.96 | 17.07 |
| | Mouse GNMT | N/A | N/A |
| | CHO MTR | 25.94 | 10.05 |
| Cells Transfected with CTH | β-Actin | 15.75 | |
| and CBS Vectors and | Mouse CTH | 19.41 | 3.66 |
| Selected in Antibiotics | Mouse CBS | 17.35 | 1.60 |
| without Adaptation | Mouse GNMT | 33.57 | 19.76 |
| | CHO MTR | 25.14 | 9.39 |
| Cells Transfected with CTH | β-Actin | 15.81 | |
| and CBS Vectors and | Mouse CTH | 18.51 | 2.41 |
| Selected in Antibiotics | Mouse CBS | 17.51 | 1.62 |
| Followed by Adaptation | Mouse GNMT | 32.97 | 19.01 |
| | CHO MTR | 25.57 | 9.66 |
| Cells Transfected with CTH, | β-Actin | 15.65 | |
| CBS, and GNMT Vectors and | Mouse CTH | 20.96 | 5.31 |
| Selected in Antibiotics | Mouse CBS | 18.28 | 2.63 |
| Without Adaptation | Mouse GNMT | 17.42 | 1.77 |
| | CHO MTR | 23.25 | 7.60 |
| Cells Transfected with CTH, | β-Actin | 15.98 | |
| CBS, and GNMT Vectors and | Mouse CTH | 19.49 | 3.48 |
| Selected in Antibiotics | Mouse CBS | 18.93 | 2.92 |
| Followed by Adaptation | Mouse GNMT | 13.49 | −2.46 |
| | CHO MTR | 25.96 | 9.95 |

### Example 7: Experiment to Adapt CBS+CTH and GNMT Cell Pools to Cysteine Free and Homocysteine Free Conditions

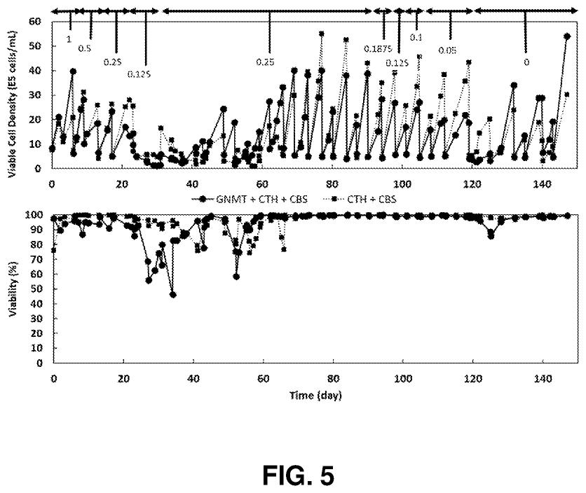

GNMT and CBS+CTH pools as described in Example 6 were cultivated in reducing concentrations of homocysteine in cysteine-free CD CHO medium to adapt the pools to cysteine free and homocysteine free conditions (FIG. 5). Adaptation was started with 2 mM homocysteine in cysteine free environment and reduced to lower concentrations in stepwise manner including 1 mM, 0.5 mM, 0.25 mM, 0.1875, 0.125 mM, 0.1, 0.05 mM and 0 mM. At each concentration levels, cells were cultivated either for few passages if the viability remained high or if the viability dropped upon transfer to the new concentration, cells were cultivated at that concentration (or a slightly elevated concentration) until the viability recovered and growth rate improved. Beta mercaptoethanol (BME) was supplemented throughout the adaptation process.

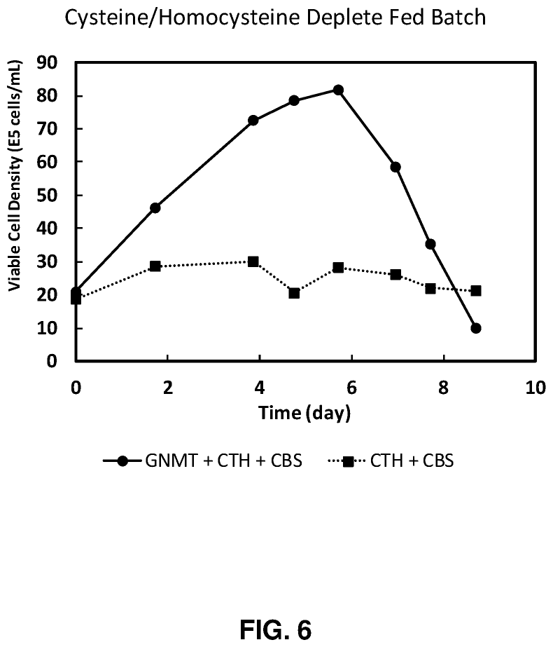

Next, GNMT and CBS+CTH cells completely adapted to cysteine-free and homocysteine-free conditions were tested for growth in fed batch cultures with BME supplementation (FIG. 6). GNMT cells (GNMT+CTH+CBS) grew to higher cell densities than the CBS+CTH cells.

### Example 8: Experiment to Assess the Growth of GNMT and CBS+CTH Cell Pools in Various Cysteine-Free Media

### Goal

The goal of this experiment was to assess the growth of cells overexpressing GNMT, CBS, and CTH genes (“GNMT cells”) and cells overexpressing CBS and CTH genes (“CBS+CTH cells”) in two different cysteine-free media. An additional goal was to assess the effect of inhibiting the activity of MTR on the growth of GNMT cells and CBS+CTH cells in cysteine-free media.

### Methods

GNMT cell pools and CBS+CTH cell pools that had been adapted to grown in cysteine-free and homocysteine-free conditions as described in Example 7 were cultivated in cysteine-free CD CHO medium or cysteine-free Pfizer internal medium. Pfizer internal medium is a chemically defined, protein-free, amino acid fortified version of DMEM:F12 medium with adjusted levels of vitamins, trace elements, sodium bicarbonate and potassium chloride, and containing polyvinyl alcohol. CD CHO medium is a commercial, chemically defined medium with proprietary composition.

GNMT cell pools and CBS+CTH cell pools were also cultivated in internal medium supplemented with different concentrations sodium nitroprusside (SNP). SNP has been established as an inhibitor of 5-methyltetrahydrofolate-homocysteine methyltransferase (MTR) enzyme (also known as methionine synthase) [Nicolaou et al. The inactivation of methionine synthase in isolated rat hepatocytes by sodium nitroprusside. Eur J Biochem. 1997 Mar. 15; 244(3):876-82]. MTR converts homocysteine back to methionine thereby decreasing metabolic flux towards production of cysteine through the transsulfuration pathway.

50 uM beta mercaptoethanol (BME) was supplemented to all the conditions.

### Results

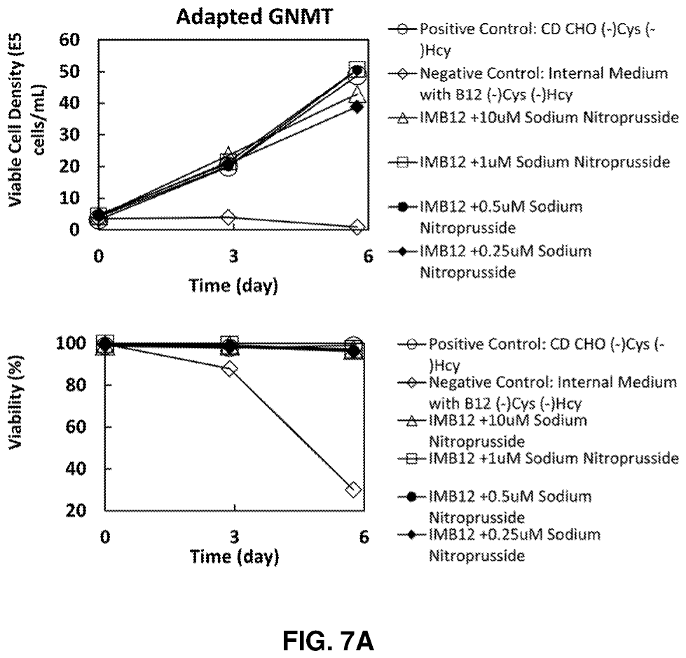

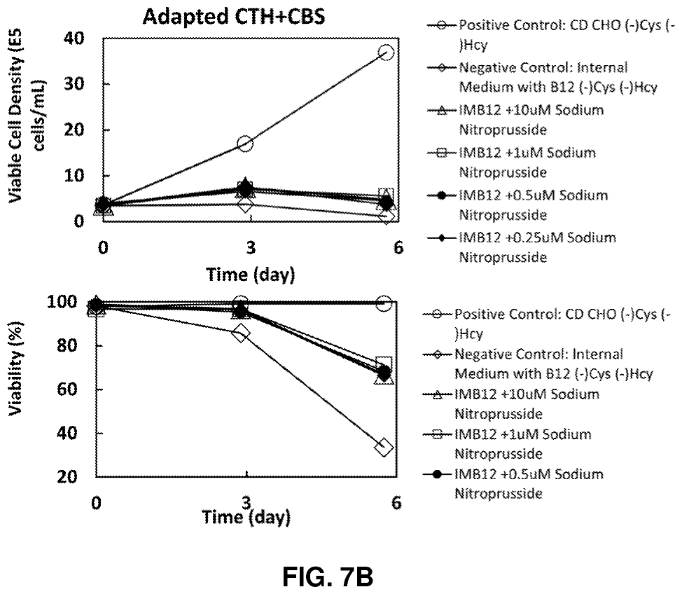

GNMT cells grew well in CD-CHO medium, but their growth was significantly impacted in the cysteine free Pfizer internal medium. Supplementation of SNP appears to restore the growth GNMT cells in the cysteine free Pfizer internal medium (FIG. 7A).

CBS+CTH cells grew well in the CD-CHO medium but not in the cysteine free Pfizer internal medium. SNP supplementation improved the cell growth marginally but didn't completely restore it (FIG. 7B).

The data suggests that inhibition of MTR enzyme helps growth of GNMT and CBS+CTH cell pools in certain cysteine free culture conditions.

### Example 9: Experiment to Test Adaptation of CBS+CTH and GNMT Pools to Beta Mercaptoethanol (BME) Free Conditions

### Goal

The goal of this experiment was to test ability of cells overexpressing GNMT, CBS, and CTH genes (“GNMT cells”) and cells overexpressing CBS and CTH genes (“CBS+CTH cells”) to grow in the absence of beta mercaptoethanol (BME) and, if either pool could grow in the absence of BME, to adapt the pool(s) to have improved growth in the absence of BME.

Glutathione (GSH) acts as a reducing agent in mammalian cells and requires cysteine to be produced. When the GNMT and CBS+CTH cell pools are cultivated in cysteine-free media, there may not be enough cysteine substrate to make GSH. This can cause cell death due to ferroptosis. Adding BME to the cell culture media fortifies the redox environment with reducing until, presumably, the cells can produce enough cysteine to support GSH production. Thus, the purpose of this experiment was to assess if cells overexpressing GNMT, CBS, and CTH genes (“GNMT cells”) and cells overexpressing CBS and CTH genes (“CBS+CTH cells”) grow in the absence of BME and if cells could be adapted to grow in the absence of BME.

### Methods

GNMT cell pools and CBS+CTH cell pools that had been adapted to grown in cysteine-free and homocysteine-free conditions as described in Example 7 were cultivated in 3-day or 4-day sequential batch cultures in cysteine-free CD-CHO media with or without 50 uM BME with the aim of testing the growth and adapting the cells to BME free conditions.

### Results

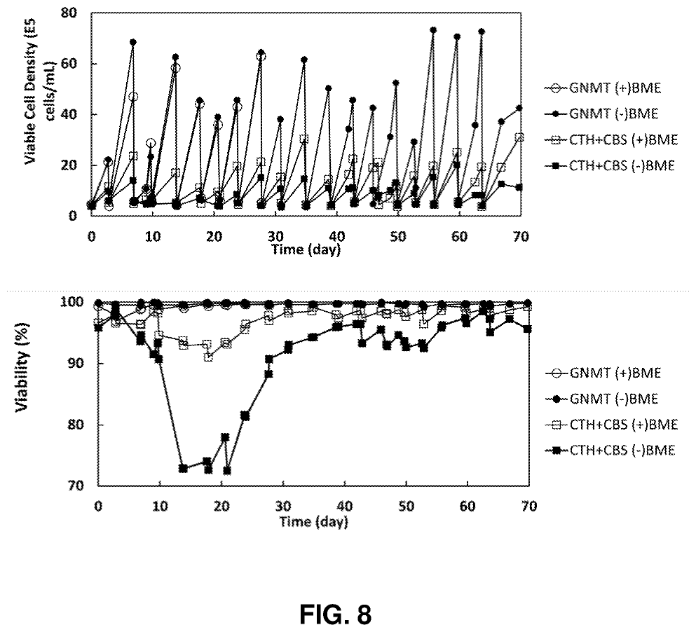

Results of the adaptation process are shown in FIG. 8. GNMT cell pool cultivated in BME free conditions did not require an adaptation period. Within one passage this pool's cell culture performance, in terms of growth and viability, were comparable to cell performance of the pool with BME. However, CBS+CTH cell pool had significantly different growth and viabilities between BME-free and BME containing conditions. This cell pool was never able to fully adapt to BME-free conditions.

Presumably, cells that are only transfected with CTH and CBS orthologs do not produce enough cysteine to regulate their redox environment whereas cells that are transfected with CTH, CBS, and GNMT orthologs are able to produce sufficient levels of cysteine. BME supplementation could be helping prevent ferroptosis in CBS+CTH cells.

### Example 10: Experiment to Isolate Single Cell Clone Selection and Analysis of Gene Expression of Genes that Confer Cysteine Prototrophy

### Goal

The goal of this experiment was to isolate single cell clones from a cell pool transfected with copies of the polynucleotide(s) containing the CBS, CTH and GNMT genes. A second goal was to evaluate the expression levels of the CBS, CTH, and GNMT genes within the single cell clones.

### Materials and Methods

The BME-free GNMT cell pools from Example 9 were thawed from a vial, cultured in a shake flask, and plated into six 96-well plates, targeting one cell per well. Three plates contained a mixture of cysteine-free conditioned media and fresh CD-CHO media at a ratio of 35:65 respectively. Another three plates contained the same media as the first three plates with the addition of 25 uM of BME. Conditioned media, also referred to as “spent media”, was obtained by centrifuging down a sample of culture containing the transfected cell pool and saving the cell-free supernatant.

After about 3 weeks, culture wells that were observed to show signs of single clonal population growth were transferred to new wells in a 24 well plate with 1 mL working volume containing Cysteine-free CD-CHO. The cells that grew in the BME containing media were supplemented with BME at this stage as well.

After another 6 days, culture wells that were identified to have achieved a desired level of growth were scaled up to new wells in 6 well plates with 3 mL working volume with the same media conditions as the previous stage. After another 6 days, culture wells that were identified to have achieved a desired level of growth were scaled up to 125 mL shake flasks with the same media conditions as the previous stage with approximately 15 mL working volume and passaged until the desired cell density was achieved for cell banking and RNA sampling.

Expression levels of mouse CBS, CTH, and GNMT transgenes and CHO MTR were obtained via RT qPCR method.

### Results

A total of 18 clones were isolated from limited dilution cloning. Levels of CHO MTR and mouse GNMT, CTH and CBS were tabulated in Table 6.

TABLE 6
RT qPCR analysis of expression levels of 18 cell clones
isolated from cell pools transfected with the CTH,
CBS and GNMT genes and selected using antibiotics,
with adaptation to L-Homocysteine depleted conditions

Gene ΔCt

| Clone | CHO | Mouse | Mouse | Mouse |
|---|---|---|---|---|
| number | MTR | GNMT | CTH | CBS |
| 1 | 9.14 | 0.28 | 4.3 | 2.75 |
| 2 | 9.01 | 0.28 | 5.01 | 2.91 |
| 3 | 8.64 | −0.36 | 4.58 | 2.73 |
| 4 | 8.91 | −0.07 | 4.21 | 3.2 |
| 5 | 8.96 | 0.03 | 5.06 | 3.14 |
| 6 | 9.18 | 0.07 | 5.22 | 2.94 |
| 7 | 10.67 | 1.05 | 5.86 | 3.86 |
| 8 | 9.34 | 0.7 | 4.65 | 3.12 |
| 9 | 9.96 | 0.83 | 5.28 | 3.19 |
| 10 | 9.28 | 1.82 | 5.48 | 3.77 |
| 11 | 9.49 | 0.38 | 4.91 | 3 |
| 12 | 9.61 | 1.04 | 5.24 | 3.17 |
| 13 | 9.59 | −0.28 | 4.59 | 2.72 |
| 15 | 9.43 | 0.19 | 5.34 | 3.82 |
| 16 | 9.13 | −0.21 | 4.45 | 2.66 |
| 17 | 9.5 | −0.39 | 4.46 | 3.09 |
| 18 | 9.35 | 0.82 | 4.66 | 3.15 |
| 19 | 9.11 | −0.46 | 4.72 | 2.65 |

### Example 11: Experiment to Test Growth and Productivity of 6 GNMT Clones and CBS+CTH Cell Pool in pH Adjusted Fedbatch Cultures

### Goal

This experiment was performed to analyze the cell growth and production performance of 6 clones isolated from a heterogenous cell pool containing mouse orthologs of CBS, CTH, and GNMT. Of the 6 clones, 3 clones had the higher expression levels of CBS, CTH, and GNMT and the other 3 clones has lower expression levels to CBS, CTH and GNMT.

### Methods

Various single cell clones from Example 10 were used. The single cell clones overexpressing CTH, CBS, and GNMT were cultivated in cysteine-free CD-CHO medium with no BME supplementation. The CBS+CTH pool was cultivated in cysteine-free CD-CHO medium in presence or absence of 50 uM of BME. The transfection control without DNA was cultivated in CD-CHO medium containing cysteine.

Production shake flasks were inoculated for 6 GNMT clones and CBS+CTH pool (with BME) from seed culture at 0.5 e6c/mL in cysteine-free CD-CHO medium. The cultures were fed based on cell growth daily from Day 3 onwards with Pfizer proprietary feed medium. pH was adjusted daily to 7.1 with a titrant. BME was supplemented only to the CBS+CTH condition once every 2 or 3 days.

### Results

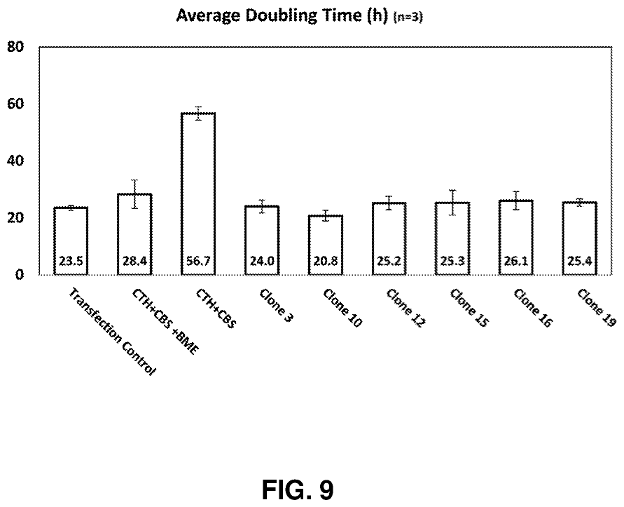

FIG. 9 shows the average doubling times and standard deviation of n=3 exemplary seed expansion passages for the GNMT clones, the wild type cell line and the CTH+CBS pool with and without BME. The transfection control and the CTH+CBS pool cultivated in the absence of BME were cultured during a different experiment from the clones and the CTH+CBS with BME. The average doubling times of these cells are shown together in FIG. 9 for comparison. Similar growth rates were observed across all conditions tested except for the CTH+CBS pool cultivated in absence of BME. The transfection control was cultured in CD-CHO containing cysteine.

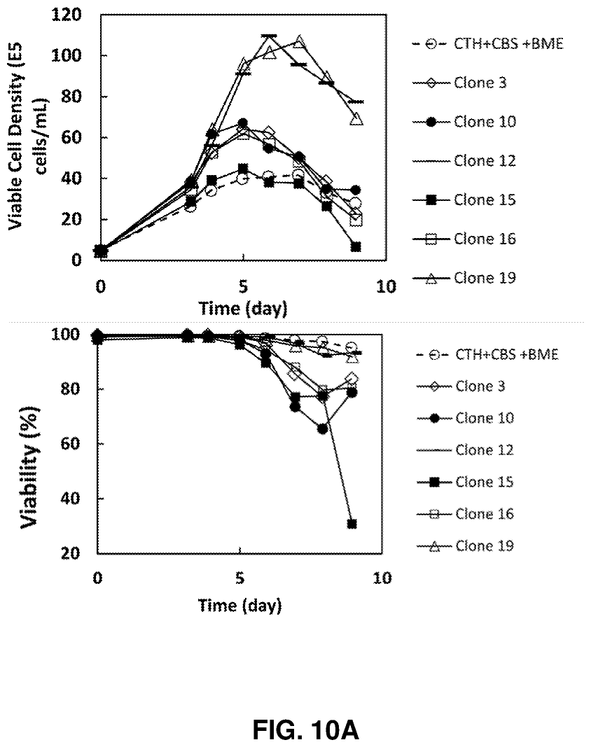

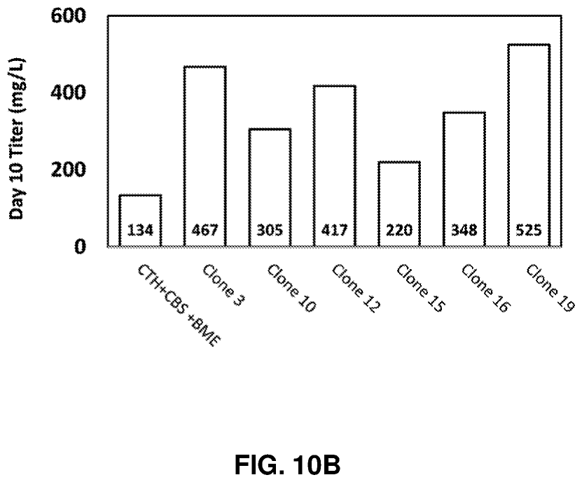

FIG. 10A and FIG. 10B shows growth, viability, and harvest titer levels of the 6 GNMT clones and the CBS+CTH pool in pH adjusted fedbatch cultures. Clones 12 and 19 had higher peak viable cell densities, i.e. ˜10e6 cells/mL, and maintained higher viabilities longer compared to the other clones. CBS+CTH condition only grew to 4e6 cells/mL but maintained higher viabilities, potentially due to supplementation of BME. All clones and the CBS+CTH pool produced product of interest (mAb). BME could be supporting higher viabilities by preventing ferroptosis caused by lipid oxidation mediated by iron in absence of sufficient levels antioxidant, such as glutathione. CBS+CTH cells could have lower levels of glutathione due to potentially reduced level of cysteine synthesis. Cysteine is a precursor for glutathione synthesis.

### Example 12: Experiment to Evaluate the Benefits of Supplementing Oleic Acid to GNMT Clones in Maintenance Cultures

### Goal

This experiment was performed to evaluate the impact of oleic acid (OA) supplementation on cell growth and cell viability of two GNMT clones. Cell death in cysteine-free conditions is reported to be caused by iron-dependent oxidation of polyunsaturated fatty acids (PUFAs) in lipid bilayer, also referred to as ferroptosis. Monounsaturated fatty acids (MUFAs) such as Oleic Acid (OA), have been shown to mitigate ferroptosis by displacing PUFAs from the phospholipids preventing accumulation of oxidized PUFAs.

### Methods

Two GNMT clones, clone 9 and 16, were cultivated for two passages in cysteine-free CD-CHO medium with and without oleic acid. In the oleic acid supplemented conditions, first passage received 1 micromolar (uM) of oleic acid and the second passage received 10 uM of oleic acid. In the third passage, the clones were cultivated in absence of OA but growth in presence of ferroptosis inducers, iron (12 uM ferrous sulphate) and zinc (10 uM zinc sulfate) to understand impact of prior culture in OA on ferroptosis.

### Results

In the third passage, growth, and viability for clone 16 expanded in absence or presence of OA was lower in iron and zinc supplemented cultures compared to the untreated conditions. However, the OA exposed conditions had higher growth and viabilities compared to the unexposed conditions. Interestingly, the benefit of exposure to OA was not observed in clone 9 (Table 7 and Table 8). This suggested that growth and viability GNMT clones may benefit from oleic acid supplementation across maintenance cultures.

TABLE 7
Viable Cell Density data for two GNMT clones at
three sample points in a batch shake flask culture.
Viable Cell Density (E5 c/mL)

| | | | Day | Day | Day |
|---|---|---|---|---|---|
| Condition | | Clone | 0 | 3 | 4 |
| No OA | CD CHO | Clone | 5.1 | 18.95 | 35.16 |
| | CD CHO + 12 uM FeSO4 | 16 | 4.02 | 14.07 | 31.09 |
| | CD CHO + 10 uM ZnSO4 | | 4.31 | 12.27 | 23.99 |
| +10 uM | CD CHO | | 4.44 | 22.52 | 48.33 |
| OA | CD CHO + 12 uM FeSO4 | | 3.63 | 19.21 | 40.8 |
| | CD CHO + 10 uM ZnSO4 | | 3.84 | 13.62 | 32.19 |
| No OA | CD CHO | Clone | 3.9 | 22.74 | 41.43 |
| | CD CHO + 12 uM FeSO4 | 9 | 3.54 | 19.65 | 39.74 |
| | CD CHO + 10 uM ZnSO4 | | 3.55 | 17.26 | 32.29 |
| +10 uM | CD CHO | | 3.85 | 21.99 | 42.9 |
| OA | CD CHO + 12 uM FeSO4 | | 3.45 | 21.94 | 38.38 |
| | CD CHO + 10 uM ZnSO4 | | 3.11 | 16.78 | 30.16 |

TABLE 8
Cell Viability data for two GNMT clones at
three sample points in a batch shake flask culture.
Viability (%)

| | | | Day | Day | Day |
|---|---|---|---|---|---|
| Condition | | Clone | 0 | 3 | 4 |
| No OA | CD CHO | Clone | 99.5 | 98.9 | 99.1 |
| | CD CHO + 12 uM FeSO4 | 16 | 98.7 | 98.5 | 99.1 |
| | CD CHO + 10 uM ZnSO4 | | 99.2 | 94.9 | 94.1 |
| +10 uM | CD CHO | | 99.1 | 98.9 | 99.3 |
| OA | CD CHO + 12 uM FeSO4 | | 99.6 | 98.6 | 99.2 |
| | CD CHO + 10 uM ZnSO4 | | 99.6 | 96.2 | 96 |
| No OA | CD CHO | Clone | 99.8 | 98.8 | 99.3 |
| | CD CHO + 12 uM FeSO4 | 9 | 100 | 99.2 | 99.3 |
| | CD CHO + 10 uM ZnSO4 | | 100 | 97.9 | 96.7 |
| +10 uM | CD CHO | | 100 | 98.8 | 99.3 |
| OA | CD CHO + 12 uM FeSO4 | | 99.6 | 99.2 | 99.1 |
| | CD CHO + 10 uM ZnSO4 | | 98.9 | 97.7 | 96.4 |

### Example 13: Experiment to Evaluate the Benefits of Supplementing Oleic Acid (OA) and Ferroptosis Inhibitors, Ferrostatin 1 (Fer1) or Vitamin K1, on Growth and Viability of GNMT Clones in Fedbatch Cultures

### Goal

This experiment was performed to evaluate the impact of oleic acid (OA) or ferroptosis inhibitors, ferrostatin 1 (Fer1) or Vitamin K1, supplementation on cell growth and cell viability of two GNMT clones. Cell death in cysteine-free conditions is reported to be caused by iron-dependent oxidation of polyunsaturated fatty acids (PUFAs) in lipid bilayer, also referred to as ferroptosis. Monounsaturated fatty acids (MUFAs) such as Oleic Acid (OA), have been shown to mitigate ferroptosis by displacing PUFAs from the phospholipids preventing accumulation of oxidized PUFAs. Fer1 has been shown to prevent ferroptosis by scavenging alkoxyl radicals produced during lipid peroxidation. Vitamin K1, a Vitamin K derivative, has been shown to prevent ferroptosis by preventing lipid peroxidation.

### Methods

Two GNMT clones (overexpressing CBS, CTH and GNMT), clone 15 and 19, were cultivated in pH-adjusted fedbatch cultures in multiple shake flasks. Cells were seeded at 0.5E6 cell/mL in cysteine-free internal basal medium and were fed with 1% cysteine-free, nutrient rich medium daily starting day 4. For the first set of shake flasks, cultures for each clone were treated with 1 uM of Fer1 or 10 uM of OA on days 0, 5 and 6, or left untreated. For the second set of shake flasks, cultures for each clone were treated with 10 uM of Vitamin K1 on days, 0, 4, 5 and 6. Viable cell densities and viabilities were measured on various days during the fedbatch culture.

### Results

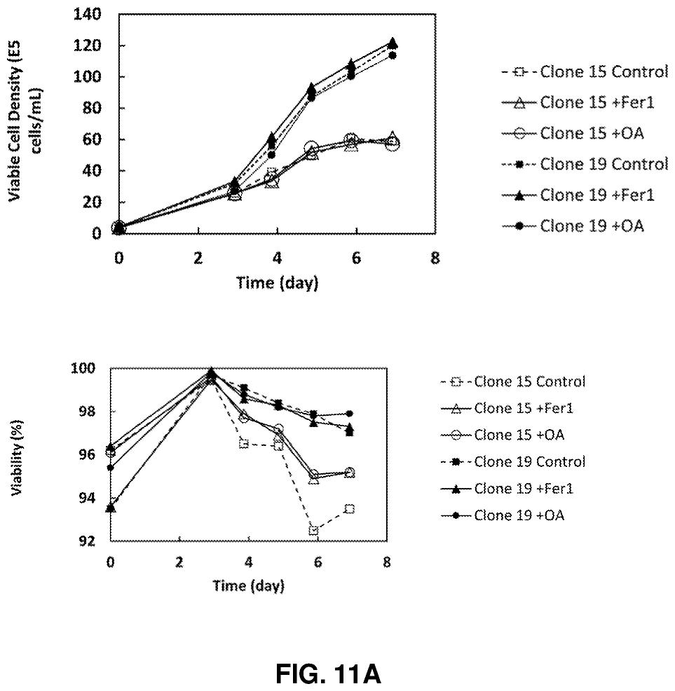

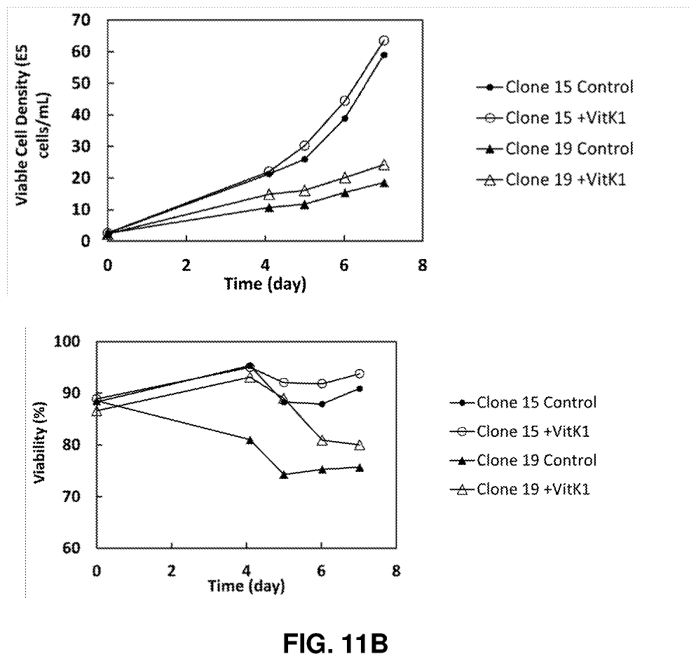

In untreated conditions, clone 19 had better growth and viabilities than clone 15 (FIG. 11A, upper and lower panels). In case of clone 15, all the conditions had similar viable cell densities. Whereas there was a difference in the viabilities observed between the treated and untreated conditions by the end of the culture. Fer1 and OA treated conditions has slightly higher viabilities than untreated conditions. In case of clone 19, all the conditions had similar viable cell densities and viabilities. Clone 15 could be producing lower levels of cysteine compared to clone 19 which could be the cause for lower growth and viabilities observed in clone 15. Supplementation of Fer1 or OA treatment could be suppressing ferroptosis in clone 15 reducing the loss of viability observed in the later stages of the culture.

In the second experimental set, the Vitamin K1 supplemented conditions demonstrated improved growth and viability for both clones (FIG. 11B, upper and lower panels).

These data suggested that growth and viability GNMT clones may benefit from oleic acid and/or Fer1 supplementation and/or Vitamin K1 supplementation across fedbatch cultures.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, textbooks, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A host cell comprising an exogenous cystathionine beta-synthase (CBS) gene and an exogenous cystathionase (cystathionine gamma-lyase) (CTH) gene.

2. The host cell of claim 1, further comprising an exogenous glycine N-methyltransferase (GNMT) gene.

3. The host cell of claim 1, wherein at least one of the exogenous CBS gene and exogenous CTH gene is stably integrated into a chromosome of the host cell.

4. The host cell of claim 2, wherein the exogenous CBS gene, exogenous CTH gene, and exogenous GNMT gene are each stably integrated into a chromosome of the host cell.

5. The host cell of claim 1, wherein the host cell is a mammalian cell and optionally wherein the mammalian cell is a mouse cell, a human cell, or a Chinese Hamster Ovary (CHO) cell.

6. (canceled)

7. A method of producing a recombinant polypeptide, the method comprising expression of the recombinant polypeptide by the host cell of claim 1, and purification of the recombinant polypeptide.

8. A recombinant polypeptide produced by the host cell of claim 1.

9. The method of claim 7, wherein the recombinant polypeptide is a polypeptide of a monoclonal antibody.

10. A composition comprising a host cell of claim 1, and a cell culture medium.

11. The composition of claim 10, wherein the cell culture medium is cysteine-deficient.

12. The composition of claim 11, wherein the cell culture medium comprises less than 2 mM cysteine, less than 1 mM cysteine, less than 500 mM cysteine, less than 200 mM cysteine, less than 100 mM cysteine, less than 50 mM cysteine, less than 10 mM cysteine, or 0 mM cysteine.

13. The composition of claim 10, wherein the cell culture medium is homocysteine-deficient.

14. The composition of claim 13, wherein the cell culture medium comprises less than 2 mM homocysteine, less than 1 mM homocysteine, less than 500 mM homocysteine, less than 200 mM homocysteine, less than 100 mM homocysteine, less than 50 mM homocysteine, less than 10 mM homocysteine, or 0 mM homocysteine.

15. A method of obtaining a host cell having a greater ability to proliferate in cysteine-deficient media, the method comprising increasing the expression of the genes CBS and CTH in the host cell, wherein the host cell has greater ability to proliferate in cysteine-deficient media as compared to the ability to proliferate in cysteine-deficient media of an otherwise identical cell that does not have increased expression of CBS and CTH in the cell.

16. The method of claim 15, further comprising increasing the expression of the gene GNMT in the host cell, wherein the host cell has greater ability to proliferate in cysteine-deficient media as compared to the ability to proliferate in cysteine-deficient media of an otherwise identical cell that does not have the increased expression of CBS, CTH, and GNMT in the cell.

17. The method of claim 15, wherein increasing the expression of the genes comprises introducing exogenous copies of the respective genes into the host cell.

18. The method of claim 15, further comprising reducing the expression or activity of the methionine synthase (MTR) gene or protein in the cell.

19. The method of claim 18, wherein reducing the expression or activity of the MTR gene or protein comprising inhibiting the MTR protein with a small molecule inhibitor.

20. The method of claim 19, wherein the small molecule inhibitor is sodium nitroprusside (SNP).

21. The method of claim 15, wherein the host cell is a mammalian cell and optionally wherein the mammalian cell is a mouse cell, a human cell, or a Chinese Hamster Ovary (CHO) cell.

22. (canceled)

23. The host cell of claim 1, wherein any one or more of:
a) the CBS gene encodes a polypeptide comprising the amino acid sequence shown in SEQ ID NO: 1, or a sequence with at least 90% homology thereof,
b) the CBS gene comprises a DNA sequence shown in SEQ ID NO: 2, or a sequence with at least 90% homology thereof;
c) the CTH gene encodes a polypeptide comprising the amino acid sequence shown in SEQ ID NO: 3, or a sequence with at least 90% homology thereof,
d) the CTH gene comprises a DNA sequence shown in SEQ ID NO: 4, or a sequence with at least 90% homology thereof,
e) the GNMT gene encodes a polypeptide comprising the amino acid sequence shown in SEQ ID NO: 5, or a sequence with at least 90% homology thereof, or
f) the GNMT gene comprises a DNA sequence shown in SEQ ID NO: 6, or a sequence with at least 90% homology thereof.
